(19) United States
(12) Patent Application Publication
(10) Pub. No.: US 2023/0374112 A1
(43) Pub. Date: Nov. 23, 2023

(54) Bioactive Collagen Peptides, Method Of Production Thereof, And Use Thereof

(71) Applicant: CORPORATION GENACOL CANADA INC., Québec (CA)

(72) Inventor: Guy Michaud, Québec (CA)

(21) Appl. No.: 18/029,833

(22) PCT Filed: Oct. 1, 2021
(86) PCT No.: PCT/CA2021/051376

§ 371 Date: Mar. 31, 2023

Related U.S. Application Data
(60) Provisional application No. 63/086,633, filed on Oct. 2, 2020.

(51) Int. Cl. C07K 14/78; C07K 5/083; C07K 5/062; C07K 5/097; C07K 5/078; A61K 38/39; C12N 5/078; C12N 5/077

(52) U.S. Cl. CPC C07K 14/78; C07K 5/0806; C07K 5/06026; C07K 5/0823; C07K 5/06165; A61K 38/39; C12N 5/0643; C12N 5/0654; A61K 38/00

(57) ABSTRACT

The invention provides a composition comprising one or more bioactive peptides from collagen hydrolysate and use thereof for reducing joint and/or bone pain such as in arthritis or osteoarthritis. The invention also provides a use of the composition for inhibiting the activity and/or expression of osteoclasts, increasing the activity and/or expression of osteoblasts, or for the treatment and/or prevention of osteoclast-related diseases. The invention also provides a method for generating bioactive peptides from collagen hydrolysate in vitro.

### TECHNICAL FIELD

The technical field generally relates to bioactive peptides from collagen hydrolysate (CH) and use of said peptides for the prevention and/or treatment of arthritis, more particularly osteoarthritis (OA) and/or osteoclast-related diseases. The present invention also relates to the method of producing bioactive peptides in vitro.

### BACKGROUND

Ingestion of collagen hydrolysate (CH) products release bioactive peptides which can directly affect human health (Albenzio et al., 2017; Kumar et al., 2015; Wang et al., 2015). The digested products of orally administered collagen hydrolysates have been shown to build up in cartilage (Bello & Oesser, 2006; Oesser et al., 1999), and shown to improve and maintain cartilage (Ferraro, Anton, & Santé-Lhoutellier, 2016), often by stimulating collagen synthesis by chondrocytes (Oesser & Seifert, 2003). For peptides derived from CH to exert their bioactive functions, they must be absorbed at the level of the gastrointestinal system and the liver, after being previously digested by the stomach and small intestine. This bioavailability can vary among products and peptides and across individuals (age, sex, genetics, nutrition, microbiote, health/diseases). First pass metabolism defines the peptides that are absorbed at the level of the gastrointestinal tract, and then are subsequently biotransformed by the liver. The final fraction represents the peptides available in the blood stream that can circulate throughout the body.

As CH products become a widely accessible option for patients with osteoarthritis (OA), studies regarding the profiles of peptides and how they influence OA and bone and cartilage health are needed, especially in experimental conditions that can mimic in vivo conditions as closely as possible.

OA is the most common form of arthritis caused by the progressive destruction of joint cartilage and associated structures such as bone, synovial and fibrous joint capsule and the periarticular musculature. Risk factors include older age, sex, obesity, joint injuries, repeated stress on the joint, genetics, metabolic diseases such as diabetes or hematochromatosis, and bone deformities. Patients will suffer from pain, stiffness, tenderness, loss of flexibility, grating sensation, bone spur and/or swelling. Epidemiologic surveys estimate that 30-40% of adults have some radiographic evidence of osteoarthritis, with at least one fourth of those having moderate or severe disease. Any joint may be damaged but joints in hands, knees, hips and spine are mostly affected. There is a need for an effective treatment for the millions of people with osteoarthritis. Currently, there is no single drug that results in reversal or prevention of osteoarthritic changes. Most of the treatments are for pain relief rather than joint repair. It has been theorized that new treatments should focus on improving the health of existing joint collagen. Rheumatoid arthritis, another common form of arthritis, is an autoimmune disease in which the body's own immune system attacks the body's joints, whereas osteoarthritis is caused by mechanical wear and tear of the joints. OA is now considered a disease of the whole joint; all articular structures form a joint and play a significant role in joint health (Castañeda et al., 2017). Notably, the subchondral and underlying trabecular bone impact the onset, progression and severity of OA. Now that OA is being regarded as a “whole” joint condition, investigating joint tissues besides cartilage has become necessary. For this reason, the effect of CH on bone health, using concentrations based on bioavailable studies, merits further investigation. Therefore, the effect of CH on osteoblasts (which build bone) and osteoclasts (which degrade bone), the latter of which has been previously implicated in both OA pathogenesis (Löfvall et al., 2018) as well as bone resorption, also merits further investigation.

Collagen is a large protein. Its molecular weight is approximately 300,000 daltons. It is the main structural component in the extracellular space in the various connective tissues in animal bodies. It is found in animals exclusively. Collagen is not a uniform substance but is rather a family of proteins. There are more than 30 types of collagen, varying in structure and occurrence, the most frequent being Types I to V. In cartilage, it is mostly type II. It is not soluble in cold or hot water. It has a triple helix structure with three amino acid chains joined together, each chain containing about 1050 amino acids. All collagens are characterized by a specific amino acid composition: high content of hydroxyproline (hyp) and glycine (gly) (almost three times the amounts in other proteins), low content of sulphur containing amino acids and absence of tryptophan. This amino acid composition is responsible for the 3D conformation of collagen. Hydroxyproline is a non-essential amino acid and the major component of collagen. Hydroxyproline can be used as an indicator to determine the amount of collagen. Increased hydroxyproline levels in the urine and/or serum are normally associated with degradation of connective tissue.

The largest commercial sources of collagen are: beef and pork (skin, hides, bone), fish skins and scales. It is mostly found in the flesh and connective tissues. It is almost always present as gelatin in by-products of commercial processing.

Gelatin and other products such as collagen hydrolysates are digested in the stomach and small intestine into either peptide components or amino acids, which can then be absorbed unaltered (Schrieber et al. 2007). Collagen products are recognized as safe components of pharmaceuticals and foods by the US Food and Drug Administration (FDA) Center for Food Safety and Nutrition and was designated as “Generally Recognized As Safe” (GRAS).

Collagen Hydrolysate (CH) is made from gelatin by hydrolyzation, i.e. enzymatic digestion to hydrolyse peptide bonds of the gelatin. Selection of enzymes, time, temperature and pH enable to control digestion of the gelatin chains to a mixture of lower molecular weight chains. Collagen hydrolysate contains peptides with different chain lengths or molecular weights, which are produced during the enzymatic hydrolysis, and help promote absorption in the small intestine. Collagen hydrolysate thus can provide the building blocks necessary for the synthesis of the cartilage matrix. Collagen in its native high molecular weight form is not absorbed; only di or tri peptides and amino acids, after the protein is digested, are absorbed by the small intestine. After being absorbed, the peptides are released into the systemic circulation. In general, absorption of peptides and amino acids are greater in enzymatically hydrolyzed collagen compared to non hydrolyzed.

Commercially available collagen hydrolysates such as Peptan™ (Darling) and Fortigel™ (Gelita) have a molecular weight of 2000-5000 Da (EP 1 885 771, CA 2 854 856, U.S. Pat. No. 9,072,724, WO 2013/079373). Genacol has developed a low molecular weight collagen hydrolysate which is used for preventing and/or reducing joint pain, lateral meniscal protrusion and/or improving cartilage (PCT/CA2017/051415). Subjects need to take about 10 g/day of these collagen hydrolysates before beneficing from any improvement, such as promoting skin elasticity, suppleness and hydration. There is a need for lower dosages of collagen hydrolysates. To do so, products with lower molecular weights or alternative forms of collagen hydrolysate can be promoted, which show increased absorption of bioactive components. These collagen hydrolysates with lower molecular weight peptides could be used for limiting the cartilage thickness loss and preventing and/or reducing joint pain such as in arthritis or osteoarthritis, and/or by improvement of cartilage abrasion grade and, in the knee, by reduction of lateral meniscal protrusion in patients. This is particularly important for elder patients who often already take several pills a day and for whom the need to improve patient adherence to treatment regimens is critical. Managing the number of pills to be taken every day is a key factor for patient adherence to their treatment. A less frequent and lower dosage results in better adherence.

Generating peptides from CH rather than synthetic peptide equivalents offers several benefits. First, CH peptides most often comprise a combination of di- or tripeptides consisting of different combinations of Hyp, Gly, Pro, and Ala amino acids. However, there could be some combinations of these amino acids that are not currently identified or possibly peptides comprising other amino acids. Second, the costs of synthetic peptides are higher than simply generating peptides from CH. Some peptides are more difficult to purify (e.g. Pro-Hyp and Gly-Pro-Hyp), and therefore are more expensive to synthesize. Finally, since CHs (and CH peptides) are currently a waste product from either the cattle industry or fish farming, the use of CHs (and CH peptides) would greatly reduce global waste and have a positive environmental impact.

Previous publications have measured CH peptides from plasma, serum or animal model studies using liquid chromatography mass spectrometry (LCMS). To date, specific peptide quantification after simulated in vitro digestion and first pass metabolism have not been demonstrated in the literature.

### SUMMARY

In one aspect, there is provided a composition comprising one or more bioactive peptides from collagen hydrolysate and a pharmaceutically acceptable excipient.

In one aspect, there is provided a composition as defined herein, for use in preventing and/or reducing joint pain in a patient.

In one aspect, there is provided a use of the composition as defined herein, for preventing and/or reducing joint pain in a patient.

In one aspect, there is provided a use of the composition as described herein, for the manufacture of a medicament for preventing and/or reducing joint pain in a patient.

In one aspect, there is provided a composition as described herein, for use in the treatment and/or prevention of arthritis in a patient.

In one aspect, there is provided a use of the composition as described herein, for the treatment and/or prevention of arthritis in a patient.

In one aspect, there is provided a use of the composition as described herein, for the manufacture of a medicament for the treatment and/or prevention of arthritis in a patient.

In one aspect, there is provided a method for preventing and/or reducing joint pain in a patient, said method comprising administering the composition as described herein to said patient.

In one aspect, there is provided a method for treating and/or preventing arthritis in a patient, said method comprising administering the composition as described herein to said patient.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

FIG. 1 shows an example of a transwell system comprising a permeable membrane, apical and basolateral chambers.

FIG. 2 shows first pass metabolism peptide results for Gly-Pro, Hyp-Gly, Ala-Hyp, and Pro-Hyp, representing the peptides that are available in the blood stream.

FIG. 3 shows first pass metabolism peptide results for Gly-Pro-Hyp available in the blood stream, from CH-GL and CH-GR.

FIG. 4 shows antioxidant capacity of cell culture supernatant after first pass metabolism.

FIG. 5 shows a schematic summary of collagen hydrolysates (CHs) undergoing in vitro digestion. Digesta were subsequently filtered and freeze-dried (FD). The FD CHs were applied to a co-culture of HIEC-6/HepG2 cells to determine peptide bioavailability (%) after first pass metabolism, which determined doses of FD CH for subsequent osteoclastogenesis and osteoblastogenesis studies.

FIG. 6 shows the results of osteoclastogenesis in the presence of different CHs. Bone marrow cells were plated, and differentiation was induced with M-CSF (50 ng/mL) and two different RANKL concentrations: RANKL50 (50 ng/mL) or RANKL100 (100 ng/mL). Cells were exposed to CH for the duration of the experiment: either CH-GL (Genacol) (FIG. 6A) or CH-GR (generic) (FIG. 6B). Average number of differentiated osteoclasts (OC) was determined after plated cells were fixed and stained with tartrate-resistant acid phosphatase (TRAP) are shown. FIG. 6C shows the representative images of stained OC using RANKL50. The negative control was treated with only M-CSF (50 ng/mL) with no RANKL in which no wells showed any positively (purple) OCs. Average OC size was determined in FIGS. 6D and 6E. Data is presented as mean±SEM. For each CH and RANKL treatment, statistical significance was assessed by one-way ANOVA with Dunnett post-hoc-test to determine differences of treatment dose to respective control (*<0.05, **<0.01, ***<0.001).

FIG. 7 shows the results in the changes in OC gene expression induced by CH during RANKL-initiated osteoclast differentiation. Gene expression after CH-GL treatment (0.01, 0.05, 0.1, 0.5 mg/mL) with RANKL 50 ng/mL (FIG. 7A) and 100 ng/mL (FIG. 7B). Gene expression after CH-GR treatment (0.01, 0.05, 0.1, 0.5 mg/mL) with RANKL 50 ng/mL (FIG. 7C) and 100 ng/mL (FIG. 7C). Statistical significance assessed by one-way ANOVA with Dunnett post-hoc-test to determine differences of treatment dose to respective control (*<0.05, **<0.01, ***<0.001). Data is reported as mean±SEM.

FIG. 8 shows the result of osteoblastogenesis in the presence of different CHs. Primary osteoblasts (OBs) were plated in osteogenic medium containing β-glycerophosphate and ascorbic acid. Cells were either not treated (control) or treated with CH (CH-GL or CH-GR) at either 0.01 or 0.1 mg/mL. OBs were fixed and stained with Alkaline phosphatase (ALP), Sirius red (SR), or Alizarin red (AR). FIG. 8A shows a representative image of OBs stained with ALP; i, pixel intensity and ii, stained area was determined. FIG. 8B shows a representative image of OBs stained with SR; i, pixel intensity and ii, stained area was determined. FIG. 8C shows a representative image of OBs stained with AR; i, pixel intensity and ii, stained area was determined. Values (pixel intensity and area) are represented as mean±SEM. For each stain and CH treatment, statistical significance was assessed by one-way ANOVA with Dunnett post-hoc-test to determine differences in treatment dose to respective control (*<0.05).

FIG. 9 shows the results of the changes in OB gene expression induced by CH. Primary osteoblasts (OBs) were plated in osteogenic medium containing β-glycerophosphate and ascorbic acid. Cells were either not treated (control) or treated with CH-GL (FIG. 9A) or CH-GR (FIG. 9B) with either 0.01 or 0.1 mg/mL. Statistical significance assessed by one-way ANOVA with Dunnett post-hoc test to determine differences in treatment dose to respective control (*<0.05, **<0.01, ***<0.001). Data is reported as mean±SEM.

### SEQUENCE LISTING

This application contains a Sequence Listing in computer readable form created Sep. 30, 2021 having a size of about 6 kb. The computer readable form is incorporated herein by reference.

TABLE 1
Description of Sequences

| SEQ ID NO: | Description |
|---|---|
| 1 | RANK forward primer |
| 2 | RANK reverse primer |
| 3 | Oscar forward primer |
| 4 | Oscar reverse primer |
| 5 | Cathepsin K forward primer |
| 6 | Cathepsin K reverse primer |
| 7 | Lair-1 forward primer |
| 8 | Lair-1 reverse primer |
| 9 | NFATC1 forward primer |
| 10 | NFATC1 reverse primer |
| 11 | DC-STAMP forward primer |
| 12 | DC-STAMP reverse primer |
| 13 | Col1a1 forward primer |
| 14 | Col1a1 reverse primer |
| 15 | Alkaline phosphatase forward primer |
| 16 | Alkaline phosphatase reverse primer |
| 17 | RunX2 forward primer |
| 18 | RunX2 reverse primer |
| 19 | Osterix forward primer |
| 20 | Osterix reverse primer |
| 21 | MMP9 forward primer |
| 22 | MMP9 reverse primer |
| 23 | MMP13 forward primer |
| 24 | MMP13 reverse primer |
| 25 | Actin-B forward primer |
| 26 | Actin-B reverse primer |
| 27 | GAPDH forward primer |
| 28 | GAPDH reverse primer |

### DETAILED DESCRIPTION

In one embodiment, identification and quantification of bioactive peptides that have an impact on human health in two collagen hydrolysates are provided. The degree of absorption of the peptides was compared through an innovative cell culture system, which is more physiologically relevant compared to current published literature. Peptides would not demonstrate bioactivity at the level of the joints without absorption. The literature has primarily investigated collagen peptides using them directly on different tissues (eg. cartilage), without first accessing if they are absorbed by the small intestine and in what amount. The processing methods for obtaining CH can affect peptide composition and quantity. Furthermore, processing can affect the products in such a way that the digestion in the stomach and small intestine are affected. The quantity of bioactive peptides and absorption differ between the products for some peptides. For example, only Genacol (GL) shows that the peptide Gly-Pro-Hyp is present and absorbed into the systemic circulation compared to the other collagen GR. In addition, products with different peptide composition and profiles can demonstrate different degrees of metabolism by the liver. Pro-Hyp from the product GL is metabolised more so than GR.

In one embodiment, the present invention may provide a composition comprising or consisting of one or more bioactive peptides derived from collagen hydrolysate (CH). In one embodiment, the present invention may provide a combination of bioactive peptides derived from CH. In some embodiments, CH peptides may comprise up to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. In some embodiments, peptides derived from CH may be di- or tripeptides. A peptide of the invention can comprise any combination or permutation comprising any amino acids. A peptide of the invention can comprise any combination or permutation comprising Hyp, Gly, Pro, and/or Ala. For example, CH peptides may comprise Ala-Ala, Ala-Gly, Ala-Hyp, Ala-Pro, Gly-Gly, Gly-Hyp, Gly-Pro, Hyp-Hyp, Hyp-Pro, or Pro-Pro. Other peptides may comprise Ala-Ala-Ala, Ala-Ala-Gly, Ala-Ala-Hyp, Ala-Ala-Pro, Ala-Gly-Ala, Ala-Gly-Gly, Ala-Gly-Hyp, Ala-Gly-Pro, Ala-Hyp-Ala, Ala-Hyp-Gly, Ala-Hyp-Hyp, Ala-Hyp-Pro, Ala-Pro-Ala, Ala-Pro-Gly, Ala-Pro-Hyp, Ala-Pro-Pro, Gly-Gly-Ala, Gly-Gly-Gly, Gly-Gly-Hyp, Gly-Gly-Pro, Gly-Hyp-Ala, Gly-Hyp-Gly, Gly-Hyp-Hyp, Gly-Hyp-Pro, Gly-Pro-Ala, Gly-Pro-Gly, Gly-Pro-Hyp, Gly-Pro-Pro, Hyp-Hyp-Ala, Hyp-Hyp-Gly, Hyp-Hyp-Hyp, Hyp-Hyp-Pro, Hyp-Pro-Ala, Hyp-Pro-Gly, Hyp-Pro-Hyp, Hyp-Pro-Pro, Pro-Pro-Ala, Pro-Pro-Gly, Pro-Pro-Hyp, Pro-Pro-Pro. In other embodiment, CH peptides are obtained, such as Pro-Pro-Gly, Pro-Pro-Gly, Gly-Ala-Hyp, Ala-Cys-Ser, Glu-Asp, Gly-Gln, Leu-Hyp, Met-Leu, Phe-Pro, Pro-Gly-Leu, Pro-Leu, Ser-Gly-Pro, Ser-Hyp, Ser-Pro, Thr-Tyr, Val-Ala and Gly-Pro-Ala. In another embodiment, different combinations of the amino acids described herein, as well as other amino acids, may be obtained. It is understood that the amino acids of the peptides described herein may be read in any orientation. For example, Hyp-Gly and Gly-Hyp, or Gly-Pro-Hyp and Hyp-Pro-Gly, are considered as the same peptide.

In one embodiment, the present invention may provide a composition comprising one or more bioactive peptides derived from collagen hydrolysate (CH), said one or more bioactive peptides comprising the tripeptide Gly-Pro-Hyp.

As used herein, the term “bioactive” refers to a compound or molecule (e.g. a peptide) that exerts a biological effect on a living organism (e.g. a human, mammal, animal), tissue or cell. In some aspects, peptides derived from CH are bioactive. Certain peptides have known or presumed biological effects. For example, Pro-Hyp is primarily associated with improved cartilage health, whereas individual and combinations of AAs (Pro, Hyp, Gly) and peptides (Pro-Hyp-Gly) have not been shown to affect chondrocyte proliferation nor differentiation (Nakatani, S., et al., 2009). Other peptides such as Pro-Hyp-Gly have demonstrated chemotactic activity towards neutrophils, monocytes and fibroblasts (Iwai, K., et al., 2005). A different sequence of the same AAs (Gly-Pro-Hyp) has been suggested to be involved in platelet aggregation by being recognized by platelet glycoprotein VI (Knight, C. G., et al., 1999). This interaction is unique. Gly-Pro-Hyp rarely exists in proteins other than collagen, and glycoprotein VI is thought to be expressed solely by platelets. Furthermore, this tripeptide has been shown to inhibit the activity of dipeptidyl peptidase-4 (DPP-IV) which has been associated with diabetes (Hatanaka, T., et al., 2014). Other peptides generated from hydrolysates (Gly-Ala-Hyp and Gly-Pro-Ala) have been assessed although only Gly-Pro-Hyp showed any activity against DPP-IV. Gly-Pro-Hyp proves to be an important modulator seeing as patients diagnosed with diabetes are at an increased risk to develop arthritis. In fact, changes in lipid and glucose metabolism are thought to have an impact on cartilage and subchondral bone health which can affect the development and progression of OA although this has not been fully confirmed (Piva, S. R., et al., 2015; Louati, K., et al., 2015).

In some embodiments, the composition or combination described herein comprises a pharmaceutically acceptable excipient, diluent, carrier, gelatin, microcrystalline cellulose, silicon dioxide, vegetable magnesium stearate, magnesium stearate, caramel, Citric acid, Glycine, L-Histidine, L-Lysine, L-Methionine, L-isoleucine, leucine, L-phenylalanine, potassium sorbate, purified water, sodium benzoate, sodium citrate, Stevia, natural vanilla flavor, flavor, aroma, and/or a compound improving taste and/or odor. In some aspects, the composition or combination further comprises hyaluronic acid, amino acid reissued such as the amino acid GABA, glucosamine, melatonin, MSM, chondroitin, vitamins such as vitamin C, curcuma and/or curcumin.

In some embodiments, the composition or combination is a pharmaceutical or nutraceutical composition and is an oral dosage form. In some aspects, the composition or combination is solid (e.g. tablet or capsule), gel (e.g. gel capsule) or liquid form. In some aspects, the oral dosage form does not have a bitter taste or odor.

In some embodiments, the collagen hydrolysate is prepared from beef, cattle, pork, poultry, or fish skins or scales, preferably from beef or pork. In some aspects, the collagen is prepared from skin, hides, or bones from an animal.

In one embodiment, the invention provides use of the composition or combination described herein, wherein the use lasts more than 3 months, more preferably 6 months and even more preferably more than 6 months.

In one embodiment, the invention provides use of the composition or combination described herein, wherein the patient is older than 45, 50, 55, 60, 65, 70, 75, 80, 85, or 90 years.

In one embodiment, the invention provides use of the composition or combination described herein, wherein the patient is a woman.

CH peptides may help promote whole joint and body health. A shift in current research, medical diagnosis and treatment plans have shown that OA is a condition of the whole joint, not only of cartilage. Therefore, having multiple bioactive peptides working together on multiple tissues can have increased health benefits, rather than isolating and administering only on one peptide with limited function and benefits. In some embodiments, the composition described herein is for use in preventing and/or reducing joint pain in a patient. In some aspects, the composition or combination described herein is for use in the treatment and/or prevention of arthritis in a patient.

In one embodiment, the present invention may provide a use for the composition described herein for preventing and/or reducing joint pain in a patient. In some aspects, there is provided a use for the composition described herein for the treatment and/or prevention of arthritis in a patient.

In one embodiment, the present invention may provide a use for the composition described herein for the manufacture of a medicament for preventing and/or reducing joint pain in a patient. In some aspects, there is provided a use for the composition described herein for the manufacture of a medicament for the treatment and/or prevention of arthritis in a patient.

In one embodiment, the present invention may provide a method for preventing or reducing joint paint in a patient, said method comprising administering the composition described herein to said patient. In some aspects, there is provided a method for treating or preventing arthritis in a patient, said method comprising administering the composition described herein to said patient. The composition may be administered orally or by injection in said patient. Injection of the composition may be directly in the affected site. In some aspects, the composition may be in the form of a gel, cream, spray, or ointment and is administered topically to said patient. The composition may be administered in combination with any commonly used drugs for treating or preventing arthritis in a patient or for preventing or reducing joint paint in a patient (e.g., non-steroidal anti-inflammatory drugs [NSAIDs] or corticosteroids). In some aspects, the composition may administered at any physiologically effective dose.

In some embodiments, the patient described herein is a patient with joint paint and/or arthritis. In some aspects, the joint pain is shoulder, elbow, hand, lumbar spine, hip or knee pain. In some aspects, the arthritis is osteoarthritis. In some aspects, the patient is an animal, in particular a mammal, in particular human. The patient may be an elderly patient.

In one embodiment, the present invention may provide a use of the composition as defined herein for inhibiting the activity and/or expression of osteoclasts. As used herein, the expression “inhibiting the activity of osteoclasts” may refer to inhibiting bone and/or cartilage resorption/degradation by osteoclasts. In some cases, this includes inhibition/downregulation of genes or proteins involved in bone and/or cartilage resorption/degradation. Examples of these genes or proteins may include, but are not limited to, those specified in Table 5. As used herein, the expression “inhibiting the expression of osteoclasts” may refer to inhibiting the differentiation or development of osteoclasts (e.g., inhibition/downregulation of osteoclast differentiating genes in progenitor cells, inhibiting the number of osteoclasts, or inhibiting the size of osteoclasts), or by killing of osteoclast cells.

In one embodiment, the present invention may provide a use of the composition as defined herein for increasing the activity and/or expression of osteoblasts. As used herein, the expression “increasing the activity of osteoblasts” may refer to increasing or enhancing bone and/or cartilage formation/growth (e.g., ossification) by osteoblasts. In some cases, this includes increase/upregulation of genes or proteins involved in bone and/or cartilage formation/growth. Examples of these genes or proteins may include, but are not limited to, those specified in Table 6. As used herein, the expression “increasing the expression of osteoblasts” may refer to increasing/enhancing the differentiation or development of osteoblasts (e.g., increase/upregulation of osteoblast differentiating genes in progenitor cells).

In one embodiment, the present invention may provide a use of the composition as defined herein for the treatment and/or prevention of an osteoclast-related disease or disorder in a patient. As used herein, the expression “osteoclast-related disease or disorder” may refer to any disease or disorder that is mediated by enhanced osteoclast activity and/or expression (e.g., numbers and/or size). Such diseases or disorders may involve increased bone and/or cartilage resorption/degradation. Examples of such diseases may include but are not limited to osteoporosis, osteoarthritis, rheumatoid arthritis, Paget's Bone Disease, bone tumors, periprosthetic osteolysis, osteopetrosis, osteopenia, or osteoclastoma. In another embodiment, the present invention may provide a use of the composition as defined herein for the manufacture of a medicament for the treatment and/or prevention of an osteoclast-related disease or disorder in a patient.

In one embodiment, the present invention may provide a method for the treatment and/or prevention of an osteoclast-related disease or disorder in a patient, said method comprising administering the composition described herein to said patient.

In another embodiment, the present invention may provide a method for inhibiting the activity and/or expression of osteoclasts, or for increasing the activity and/or expression of osteoblasts. In some cases, the method includes treating osteoblasts or osteoclasts with the composition defined herein. Said method may be performed in vitro (e.g., in vitro differentiated osteoclasts or osteoblasts), ex vivo (e.g., primary cells or differentiated from harvested progenitor cells), or in vivo.

In one embodiment, the present invention may provide a method for generating peptides from collagen hydrolysate in vitro, said method comprising the steps of digesting the collagen hydrolysate in a simulated salivary fluid; digesting in a simulated gastric fluid; digesting in a simulated intestinal fluid; cooling and filtering the digesta; freeze-drying the filtrate; and determining peptide profile and content. Each step of simulated digestion can be done at room temperature, but preferably at temperatures simulating body temperatures (e.g. 37° C.). Simulated salivary fluid simulates the first step of digestion. Said salivary fluid may comprise any proteins (e.g. enzymes) or compounds commonly found in saliva. In some aspects, the simulated salivary fluid comprises α-amylase and is at a neutral pH. In some aspects, the pH of the salivary fluid is at any pH between 6 and 8, 6.5 and 7.5, or 6.8 and 7.2, or is preferably 6.9. The salivary gastric fluid simulates the gastric step of digestion. Said gastric fluid may comprise any proteins or compounds commonly found in stomach or esophagus. In some aspects, the simulated gastric fluid comprises pepsin solution at an acidic pH. In some aspects, the pH of the gastric fluid is at any pH between 1 and 7, 1 and 6, 1 and 5, 1 and 4, or 1 and 3, or is preferably 2. The simulated intestinal fluid simulates the intestinal step of digestion. Said intestinal fluid may comprise any proteins or compounds commonly found in the caecum, duodenum, small, large intestine, and/or colon. The simulated intestinal fluid may comprise a bile solution at an alkaline pH. In some aspects, the pH is between 6 and 9, 6.5. and 8.5, 7 and 8.5, or 7.5 and 8.5, or is preferably 8. In some aspects, the peptide content is determined by commonly known methods such as high-performance liquid chromatography (HPLC) or liquid-chromatography mass spectrometry (LCMS), or preferably by capillary electrophoresis (CE). Capillary electrophoresis is an instrument that is versatile, requires low cost consumables, and allows for a short method development time, as compared to HPLC or LCMS.

Other known methods for generating and studying bioactive peptides, such as certain in vivo methods, have several disadvantages compared to the in vitro method described herein. For example, CH products may be given to human subjects, ingested, and generated bioactive peptides may be isolated from the blood (Wauquier et al., 2019). Studies involving humans, however, are costly. These types of studies also depend on recruitment and the time that volunteers have. Therefore, delays are common. Using human volunteers for samples also requires ethics approval, which can delay experimental timelines, and sometimes the experimental proposal can be rejected. These experiments require rigorous regulatory measures. Furthermore, the use of human blood samples, limits the quantity of sample and timeline to complete experiments. Cell culture experiments, on the other hand, allow for more flexibility. In addition, each person has a different digestive system, which can impact bioavailability of the resulting peptides. Diet, lifestyle, and many other factors can impact the digestive system. Therefore, the resulting data can often become very variable. Cell culture uses a standardized approach, such that the experiments are highly reproducible and consistent. Additionally, the biotransformation of the peptides by the liver in humans is unknown. In contrast, in vitro methods allow for the assessment of biotransformation and permeability percentages of peptides. Dosing can be further investigated more easily in cell culture to understand the limits of effectiveness. In conclusion, in vitro methods for generating and studying CH peptides are more cost effective, accurate, and efficient.

Furthermore, generating CH and CH peptides from collagen can be more beneficial than synthesizing a limited number of specific equivalent peptides, as previously mentioned. A cocktail of peptides might be required rather than 3 or 4 peptides only. There could be several novel combinations of amino acids of CH peptides or possibly novel peptides. The costs of synthetic peptides are far greater and often due to the difficulty in purifying certain peptides. Finally, since CHs are currently a waste product from either the cattle industry or fish farming, the use of CHs would greatly reduce global waste and have a positive environmental impact.

In one embodiment, the present invention may provide a superior source of collagen hydrolysate, compared to other commercially available collagen hydrolysates (e.g. Generic CH obtained from the same source (bovine); other generic collagen could have been from other sources (porcine or fish collagen) but would have peptides differences from the start). In some aspects, Genacol CH can be more readily digested and metabolized into bioactive CH peptides, such as those described herein, as compared to its Generic counterpart. In some aspects, digesta from Genacol CH comprises novel CH peptides not found in Generic CH digesta. Novel peptides isolated from Genacol CH may be bioactive and effective in the treatment/prevention of OA or reducing/preventing joint pain in a patient.

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one” but it is also consistent with the meaning of “one or more”, “at least one”, and “one or more than one”.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, advantages and features of the present description will become more apparent upon reading of the following non-restrictive description of specific embodiments thereof, given by way of example only with reference to the accompanying drawings.

### Example 1—Materials and Methods

### In Vitro Digestion

Simulated human digestion was completed to represent salivary, gastric and intestinal digestion and provide digests for first pass metabolism studies (Alemán, Gómez-Guillén, & Montero, 2013; Miranda, Deusser, & Evers, 2013). Based on the daily dose used in the clinical trials and the current recommendations of the bottle, 1200 mg of Genacol CH product (bovine) or a generic CH product (bovine) was added (3% w/v) to sterile ddH₂O (Bernado & Azarcon, 2012; Bruyère et al., 2012; Feliciano et al., 2017). Three independent digestions for each CH product were completed. The vessels were placed in a 37° C. water bath, with continuous agitation. A pepsin solution was added, the pH adjusted to 2 and incubated for 30 min. Then, a bile solution was added, the pH adjusted to 7.5 and incubated for 90 min at 37° C. Pepsin (4% w/w protein basis) and pancreatin (4% w/w protein basis) were added (Aleman et al., 2013). The pancreatin digestion was stopped with the addition of NaOH, until the pH was 10 and the resulting digesta was cooled and frozen at −80° C. Digesta subsamples were taken and filtered using 0.2 μm syringe filters.

The final digesta was filtered using a molecular weight cut off (MWCO) of 10 kDa in a stirred Amicon™ ultrafiltration membrane reactor at 4° C. under a nitrogen gas pressure of 40 psi (Iskandar et al., 2015). The filtrates were freeze-dried at −50 to −60° C. and 0.85 mBar (0.64 mm Hg) (Gamma 1-16 LSC, Christ, Osterode am Harz, Germany) and stored at −80° C. until used in cell culture. For OC and OB studies, the filtrates were then freeze-dried at −50 to −60° C. and 0.85 mBar (0.64 mm Hg) (Gamma 1-16 LSC, Christ, Osterode am Harz, Germany) and stored at −80° C. until applied to cell culture experiments.

### Materials

Two bovine-sourced CH products were used: Original Formula® (Genacol, Blainville, QC) (CH-GL) and a generic brand (such as Selection™, Uniprix, Quebec, Canada) (CH-GR). HIEC-6 (ATCC® CRL-3266™) and Hep G2 (ATCC® HB-8065™) cells were purchased from ATCC to represent the gastrointestinal and liver wall lining, respectively. HIEC-6 cells were cultured using OptiMEM™ 1 Reduced Serum Medium (Gibco Catalog No. 31985) with 20 mM HEPES, 10 mM GlutaMAX™ (Gibco Catalog No. 35050), 10 ng/mL Epidermal Growth Factor, 4% fetal bovine serum (FBS). HepG2 cells were grown using ATCC-formulated Eagle's Minimum Essential Medium (Catalog No. 30-2003), with 10% FBS. Cells were maintained at 37° C. with 90% relative humidity (RH) and 5% C02 in culture medium as described herein.

### First Pass Metabolism

### Co-Culture

HIEC-6/HepG2 cell coculture system was used to determine the bioavailability of key bioactive peptides from CHs after digestion. HIEC-6 and HepG2 cells were cultured separately but then later combined in a transwell system. The methods are adapted from Ekbatan et al., (2018), Takenaka et al., (2016) and Takenaka et al., 2014.

HIEC-6 cells were seeded onto 24-well polyester (PET) ThinCert™ inserts from Greiner Bio-One (Kremsmünster, Austria) at a density of 1×10⁵ cells/well. The medium was changed every 2 days and the cells were grown for a total of 8-9 days. Transepithelial electrical resistance (TEER) was measured using a volt-ohmmeter to assess integrity of monolayer. Experiments were conducted when the TEER reached 100 ohm/cm² as has been shown to be appropriate for HIEC cells (Takenaka et al., 2014). Three individual plate experiments were completed for each CH product.

Preliminary studies were completed to assess for optimal peptide dose range and determined using an MTT assay, a colorimetric assay for assessing cell metabolic activity and the number of viable cells present. A concentration of 2 mg/ml was chosen as the optimal dose. At time 0, the apical medium was replaced with medium containing 2 mg/ml of freeze-dried CH dissolved in medium using either the Genacol (GL) peptide or the Generic (GR) CH products. HepG2 cells were added to the basolateral side of the transwell (1 million cells/mL). The plates were incubated for 2 h at 37° C., 5% C02. Subsamples were taken at time 0, and after 2 h. Another 3-hour incubation followed with HepG2 cells only, after the inserts containing HIEC cells were removed. Samples were also obtained after incubation from the basolateral side (5 h).

Cell culture samples were centrifuged at 2000×g for 15 min. The supernatants were collected, and a subsample used for peptide analysis and peptide permeability was calculated. The remaining supernatant, from the basolateral compartment and final timepoint, was stored at −80° C. and to be used as a treatment for bone and/or joint cultures, representing the bioactive peptides that have undergone first pass metabolism.

### Peptide Analysis

Peptide analysis was completed using a novel method using capillary electrophoresis (CE). The cell culture supernatant was filtered using Amicon® Ultra 0.5 mL Centrifugal Filters (Millipore Sigma, Cat no. UFC5010). The concentrated protein pellet was discarded, and the filtered supernatant containing the low molecular weight peptides was collected. The filtrate was diluted with a 0.1 M phosphate buffer (pH 2.5) and each replicate was injected into the CE (Lumex Instruments; Fraserview Place, BC, Capel 205M,) twice by pressure (30 mbar for 10 s) at 20° C. with a capillary of 60 cm total length, 50 cm effective length, 75 μm inside diameter. Analysis was completed at 20 kV at 205 nm for 30 min. The sensitive detection and quantitation of key peptides (Pro-Hyp, Gly-Pro-Hyp, Gly-Pro, Hyp-Gly, and Ala-Hyp) were completed using external calibration curves. A minimum of six points of calibration were used to produce standard curve and the linearity was assessed by the correlation coefficient, R². The chromatograms were processed using the software package Elforun™ (Lumex instruments).

Each treatment had 3 biological replicates for each of these replicates, 2 injections were performed. Treatments completed were: cell culture blank (CCB; cells were seeded, but medium was added as the treatment rather then a CH product), pre-digested and freeze dried Genacol CH (GL) and a Generic CH (GR).

### Osteoclasts: Isolation and Study Design

Freeze-dried CH digesta were dissolved in α-MEM with L-glutamine, without any additives, and sterile filtered (0.22 μm) before application to cell culture (OC and OB studies). Osteoclasts were obtained using previously established protocols (Boraschi-Diaz et al., 2016). In brief, femora and tibiae were isolated from C57BL/6 mice and used to collect bone marrow cells. The cell suspension was centrifuged, and the pellet was treated with red blood cell lysing buffer (Sigma Aldrich, R7757). The pellet was washed with complete culture medium [αMEM with L-glutamine (Gibco 12,000-022), 10% fetal bovine serum (FBS, Wisent 080152), 1% sodium pyruvate (Wisent 600-110-EL), 1% penicillin-streptomycin (Wisent 450-201-EL), 0.1 mg/mL ampicillin (BioShop Canada Inc AMP201.25)] and centrifuged again. The final pellet was resuspended in culture medium supplemented with Macrophage colony-stimulating factor (M-CSF 50 ng/mL) and incubated overnight in a T75 cm² flask (Falcon 353136). The following day, non adherent cells were collected and plated at a cell density of 50×10³ cells/cm² in a 48-well plate. Cells were incubated at 37° C., with 5% C02, M-CSF, and RANKL (50 or 100 ng/mL). After 3 days, cell culture medium was changed, and changed every 2 days onward. Mature OCs were observed between days 5-7 and either stained or collected for further analysis. The osteoclasts cultures were exposed to CH treatment for the duration of the experiment. For each CH supplement, four doses were assessed under two different RANKL concentrations. A negative control, CH control, and a differentiation control was also completed. A full description of control descriptions and treatments are available in Table 2.

TABLE 2
Osteoclast study design: description of controls and treatments

| | |
|---|---|
| Control | Medium supplements |

Osteoclastogenesis

| | |
|---|---|
| Negative Control | M-CSF |
| CH-GL CH control | M-CSF + CH-GL (0.01 and 0.1 mg/mL) |
| CH-GR CH control | M-CSF + CH-GR (0.01 and 0.1 mg/mL) |
| Differentiation Control 1 (RANKL50) | M-CSF + RANKL50 |
| Differentiation Control 2 (RANKL100) | M-CSF + RANKL100 |
| CH-GL | M-CSF + RANKL50 + CH-GL (0.01, 0.05, 0.1, 0.5 mg/mL) |
| | M-CSF + RANKL100 + CH-GL (0.01, 0.05, 0.1, 0.5 mg/mL) |
| CH-GR | M-CSF + RANKL50 + CH-GR (0.01, 0.05, 0.1, 0.5 mg/mL) |
| | M-CSF + RANKL100 + CH-GR (0.01, 0.05, 0.1, 0.5 mg/mL) |

Osteoblastogenesis

| | |
|---|---|
| Differentiation Control | Ascorbic acid + β-glycerophosphate |
| CH-GL | Ascorbic acid + β-glycerophosphate + CH-GL (0.01 mg/mL) |
| | Ascorbic acid + β-glycerophosphate + CH-GL (0.1 mg/mL) |
| CH-GR | Ascorbic acid + β-glycerophosphate + CH-GR (0.01 mg/mL) |
| | Ascorbic acid + β-glycerophosphate + CH-GR (0.1 mg/mL) |

Osteoclastogenesis: CHs were dissolved in α-MEM with L-glutamine, without any additives, and sterile filtered (0.22 μm) before application to cell culture. M-CSF was added to each control and treatment 50 ng/mL. RANKL application was at 50 ng/mL (RANKL50) or 100 ng/mL (RANKL100). Osteoblastogenesis: CHs were dissolved in α-MEM with L-glutamine, without any additives, and sterile filtered (0.22 μm) before application to cell culture. Ascorbic acid (50 μg/mL) and 4 mM β-glycerophosphate were added to each control and treatment. For OC and OB cells were grown in α-MEM with L-glutamine, fetal bovine serum, 1% sodium pyruvate, 1% penicillin-streptomycin, 0.1 mg/mL ampicillin.

### Osteoclast Quantification and Size Analysis

Mature OCs were fixed using 10% buffered formalin (pH 7.4) in 1×PBS for 10 min at room temperature and stained using tartrate resistant acid phosphatase (TRAP) commercial kit (Sigma 387A-KT), as outlined in Boraschi-Diaz et al., 2016. Images were obtained using a Cytation 5™ (BioTek Cytation 5 Imaging Reader, model CY5V, Winooski, Vermont, USA) and visualized and processed using Gen5™ Image Prime Software (BioTek Instruments, Version 3.09.07, Winooski, Vermont, USA). Mature OCs were counted and defined as large cells with more then 3 nuclei and positive (purple) staining. Cell size was measured using Image J™.

### Osteoblasts: Isolation and Study Design

Osteoblasts were obtained using adapted protocols from Orriss et al., (2014) [4]. In brief, femora and tibiae were isolated from mice, and bone marrow cells were isolated and used for osteoclastogenesis. Bones were washed in 70% ethanol and 1×PBS, then placed in physiological solution, where they were chopped into smaller pieces using scissors. OBs were isolated by a sequential enzyme digestion using a 4-step process (collagenase-collagenase-EDTA-collagenase “CCEC” method). Bones were incubated with solution 1 (10 mL physiological solution+125 μL 0.25% trypsin+5 μL collagenase P (100 mg/mL)) while shaking for 15 min. Solution 1 was aspirated, and solution 2 (10 mL physiological solution+125 μL 0.25% trypsin+10 μL collagenase P (100 mg/mL)) was added for 30 min. Solution 2 was aspirated and a final enzymatic solution (10 mL physiological solution+125 μL 0.25% trypsin+100 μL collagenase II (100 mg/mL)) was added to the bones for 1 h at room temperature and shaken viscously every 10 min. Afterwards, the bone pieces were plated in a 10 cm petri dish and 10 mL of cell culture medium [αMEM with L-glutamine (Gibco 12,000-022), 10% fetal bovine serum (FBS, Wisent 080152), 1% sodium pyruvate (Wisent 600-110-EL), 1% penicillin-streptomycin (Wisent 450-201-EL), 0.1 mg/mL ampicillin (BioShop Canada Inc, AMP201.25)]. Petri dishes were incubated at 37° C., with 5% C02 for 5-10 days. Medium was changed after 3 days of culture and cultures were maintained until confluent.

Once confluent, cells were collected using 0.25% trypsin, passed through a filter to remove bone pieces, and resuspended in cell culture medium. Cells were plated into 6 well-plates at a cell density of 5000 cells/cm² with cell culture medium and allowed to acclimatize. On day 3, cell medium was changed, and ascorbic acid (50 μg/mL) and 4 mM β-glycerophosphate was added alongside CH treatments (Table 2). Media with supplements were changed every other day until day 28, where cells were either stained or collected for further analysis.

### Osteoblast Analysis: Fixing and Staining

Osteoblast cultures were fixed and stained with alizarin red, alkaline phosphatase, and Sirius red as described by Orris et al., (2014) and Orris et al., (2012), as well as associated staining kit instructions. Stains each detect different characteristics of OBs (Table 3). Images were obtained using a Cytation 5 (BioTek Cytation 5 Imaging Reader, model CY5V, Winooski, Vermont USA) and visualized and processed using Gen5 Image Prime Software (BioTek Instruments, Version 3.09.07) for pixel intensity, as well as stain area (μm²).

TABLE 3
Description of OB stains and their purpose

| Stain | Characteristics/Stain detects: |
|---|---|
| Alkaline phosphatase | active osteoblasts |
| Sirius Red | deposited collagen |
| Alizarin Red | mineralization of bone nodules |

### Alizarin Red

Cells were rinsed carefully with PBS and fixed with 10% buffered formalin (pH 7.4) for 8-10 min at RT. Afterwards, the formalin was aspirated, and wells were washed again with PBS and left to air dry. Once dry, wells were rinsed with 70% EtOH, and again left to dry. Finally, Alizarin Red staining solution (1% Alizarin Red (Sigma, A5533) (w/v) in distilled water, adjusted to pH 5.5, and filtered), was added to each well and incubated for 5-15 min at RT. Once stained, wells were washed 3 times with 50% EtOH, followed by distilled water and PBS.

### Alkaline Phosphatase

Cells were fixed as described above. The staining solution was made by mixing Solution A (3.75 ml of milliQ water and 0.2M Tris-HCL (pH 8.3) with 4.5 mg of Fast Red Violet Salt (Signma F3381)) with Solution B (0.75 mg Naphthol (Sigma N5000) and 30 μl of N,N-Dimenthyformamid (Fisher Scientific BP1160)) and filtered (0.22 μm) to remove any precipitate. The staining solution was added to each fixed and dry well for 8-15 min at RT in the dark. Afterwards, the staining solution was aspirated, and the wells washed with distilled water.

### Sirius Red

Cells were rinsed carefully with PBS and fixed with ice-cold 70% EtOH for 1 h at 4° C. Afterwards, the EtOH was aspirated, and the wells were washed again with PBS. Plates were left to air dry at RT. Once dry, wells were stained using the Picro-Sirius Red Stain Kit (Abcam, ab150681). In brief, the Sirius red staining solution was added to the wells and incubated for 1 h on a shaker. Following this, the wells were washed twice with the acetic acid solution from the staining kit, then washed with milliQ water and allowed to air dry.

Quantitative Real-Time PCR (cIPCR)

RNA was isolated from OC and OB cultures samples using TRIzol™ (#15596026, ThermoFisher Scientific, Waltham, USA) according to the manufacturer's instructions. Reverse transcription was performed using the High-Capacity cDNA Reverse Transcription Kit (#4368813, Applied Biosystems™, ThermoFisher Scientific, Waltham, USA). Real-time PCR was completed using a QuantStudio™ 7 Flex System (ThermoFisher Scientific, Version 1.3, Waltham, USA), SYBR™ Green PCR Select Master Mix (Fisher Scientific, 4472918) and primers (summarized in Table 4). Gene expression was analyzed using actin and glyceraldehyde 3-phosphate dehydrogenase (GAPDH) as endogenous controls and an internal plate control was used to compare PCR plates to each other. Gene expression was analyzed according to the delta-delta Ct method. OC data was logged to better demonstrate gene expression data.

TABLE 4
Primer list for qPCR analysis

NCBI Reference

| | | |
|---|---|---|
| Gene | Sequence ′5-′3 | Sequence |

Osteoclasts

| | | | |
|---|---|---|---|
| RANK | Forward | GCATCCCTTGCAGCTCAACA (SEQ ID NO: 1) | NM_009399.4 |
| | Reverse | ATGGAAGAGCTGCAGACCAC (SEQ ID NO: 2) | |
| Oscar | Forward | TCGCTGATACTCCAGCTGTC (SEQ ID NO: 3) | NM_175632.3 |
| | Reverse | TCTGGGGAGCTGATCCGTTA (SEQ ID NO: 4) | |
| Cathepsin K | Forward | CAGTAGCCACGCTTCCTATCC (SEQ ID NO: 5) | NM_007802.4 |
| | Reverse | ACGCCGAGAGATTTCATCCA (SEQ ID NO: 6) | |
| Lair-1 | Forward | CTGTACCCCTGGGCAACTTT (SEQ ID NO: 7) | NM_001302681.1 |
| | Reverse | TTCCATAAAGGTGCTGCCGT (SEQ ID NO: 8) | |
| NFATC1 | Forward | CCCGGAGTTCGACTTCGATT (SEQ ID NO: 9) | NM_016791.4 |
| | Reverse | TCTCTGTAGGCTTCCAGGCT (SEQ ID NO: 10) | |
| DC-STAMP | Forward | TTTCCACGAAGCCCTAGCTG (SEQ ID NO: 11) | NM_029422.4 |
| | Reverse | GCGTTCCTACCTTCACGGAG (SEQ ID NO: 12) | |

Osteoblasts

| | | | |
|---|---|---|---|
| Col1a1 | Forward | GAGCGGAGAGTACTGGATCG (SEQ ID NO: 13) | NM_007742. |
| | Reverse | GTTCGGGCTGATGTACCAGT (SEQ ID NO: 14) | |
| Alkaline | Forward | CAGGCCGCCTTCATAAGCA (SEQ ID NO: 15) | NM_007431.3 |
| phosphatase | Reverse | GTGCCGATGGCCAGTACTAA (SEQ ID NO: 16) | |
| RunX2 | Forward | GCTTCTCAGCTTTAGCGTCG (SEQ ID NO: 17) | NM_001145920.2 |
| | Reverse | AAGGTGCCGGGAGGTAAGT (SEQ ID NO: 18) | |
| Osterix | Forward | GATGGCGTCCTCTCTGCTTG (SEQ ID NO: 19) | NM_130458.4 |
| | Reverse | GGGCTGAAAGGTCAGCGTAT (SEQ ID NO: 20) | |
| MMP9 | Forward | CCAGCCGACTTTTGTGGTCT (SEQ ID NO: 21) | NM_013599.4 |
| | Reverse | TGGCCTTTAGTGTCTGGCTG (SEQ ID NO: 22) | |
| MMP13 | Forward | GCCATTACCAGTCTCCGAGG (SEQ ID NO: 23) | NM_008607.2 |
| | Reverse | GGTCACGGGATGGATGTTCA (SEQ ID NO: 24) | |

Housekeeping Genes (endogenous controls)

| | | | |
|---|---|---|---|
| Actin-B | Forward | TGTTACCAACTGGGACGACA (SEQ ID NO: 25) | NM_007393.5 |
| | Reverse | GGGGTGTTGAAGGTCTCAAA (SEQ ID NO: 26) | |
| GAPDH | Forward | ACCCAGAAGACTGTGGATGG (SEQ ID NO: 27) | NM_001289726.1 |
| | Reverse | CACATTGGGGGTAGGAACAC (SEQ ID NO: 28) | |

Genes were selected based on function characteristics (Tables 5 and 6).

TABLE 5
Summary of osteoclast genes: functions

| Osteoclast Genes | Main Role |
|---|---|
| RANK | differentiation |
| NFATC1 | differentiation |
| DC-Stamp | cell fusion |
| OSCAR | differentiation |
| Lair-1 | collagen binding receptor |
| Cathepsin K | cleaves & removes type 1 collagen fibers |
| β-actin | housekeeping |
| GAPDH | |

TABLE 6
Summary of osteoblast genes: functions

| Osteoblast Genes | Main Role |
|---|---|
| Alk Phosphatase | Mineralization (calcium & |
| | phosphorous) |
| RunX2 | Differentiation |
| Col1a1 | Makes collagen (type 1) |
| Osterix | Differentiation (& role in chondrocyte |
| | differentiation) |
| MMP9 | Degradation of extracellular matrix |
| MMP13 | Facilitate breakdown of extracellular |
| | proteins |
| β-actin | housekeeping |
| GAPDH | |

### Example 2—First Pass Metabolism of Collagen Hydrolysates (CH)

The capillary electrophoresis (CE) methodology mentioned above was used to identify and quantify specific CH peptides from cell culture, after undergoing first pass metabolism. This method presented with some challenges, seeing as there was a lot of cellular debris, proteins and other compounds found in the cell culture medium, which are necessary for the cells to grow, but can interfere with analysis. The peptide content measured reflects what is available in the blood stream, which can travel to other major parts of the body to exert their bioactivity. This novel combination of cells and method of analyzing the first pass metabolism of these peptides using CE has never been reported.

The capillary electrophoresis is an instrument that is versatile, low cost consumables, and allowed for a short method development time.

### Results

### Peptide Quantification

Peptides Ala-Hyp (AH), Pro-Hyp (PH), Hyp-Gly (HG), Gly-Pro (GP), and Gly-Pro-Hyp (GPH) were assessed. No peptide detection was observed for the cell culture controls for each replicate and for each timepoint, where medium was added to the apical side rather than the CH peptide treatments. This ensures that there are no background bioactive peptides that are found within our experimental set up and media used.

The peptide Pro-Hyp-Gly was not included as part of the analysis of first pass metabolism because the initial contents of Pro-Hyp-Gly after in vitro digestion were not statistically different between Genacol and Generic CH treatments, and therefore a significant difference after absorbance was not expected.

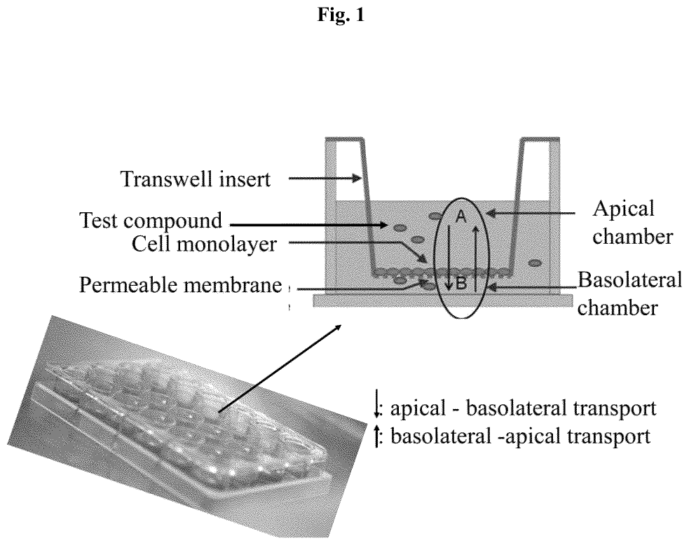

The peptide content from the two chambers of the transwell were measured: apical and basolateral side (FIG. 1). Apical represents the intestinal lumen whereas the basolateral side has the hepatic cell line, which represents the hepatic metabolism of the peptides, and simulates the peptide circulating in the blood stream.

Timepoints during the co-culture/first pass metabolism include TO, T2, and T5. TO (time 0) represents the initial dose of peptides in the apical compartment, and the potential background peptide concentration in the basolateral side. T2 represents the sample timepoint after 2 h, from apical side and basolateral side. After this timepoint, the insert with intestinal cells was discarded, as the phase for intestinal absorption has been completed. The cultures continue to incubate with only liver cells to reflect the potential metabolism of the peptides. Finally, T5 represents the sample timepoint after 5 h (only basolateral). This measured hepatic metabolism of the peptides, and the final concentration of peptides expected to be circulating in the blood stream.

Table 7 represents the quantified peptide results from first pass metabolism with digested Genacol (GL) CH and Generic (GR) CH, obtained using CE.

TABLE 7
Peptide concentration after first pass metabolism of Genacol
or Generic CH, at every timepoint for the apical compartment.

| | Apical T0 | | Apical T2 | |
|---|---|---|---|---|
| Treatment | GL | GR | GL | GR |
| Gly-Pro (μg/ml) | 37.92 ± 5.48 | 36.21 ± 2.12 | 9.792 ± 0.401 | 11.25 ± 0.90 |
| Hyp-Gly (μg/ml) | 0.7561 ± 0.2822 | 0.5469 ± 0.1594 | 0.1497 ± 0.0017 | 0.1919 ± 0.0393 |
| Ala-Hyp (μg/ml) | 19.69 ± 1.04 | 10.91 ± 6.32 | 0.3081 ± 0.0748 | 3.630 ± 0.180* |
| Pro-Hyp (μg/ml) | 10.32 ± 1.50 | 11.54 ± 1.22 | 3.520 ± 0.080 | 4.108 ± 0.303 |
| Gly-Pro-Hyp (μg/ml) | 2.030 ± 0.033 | 1.780 ± 0.336 | 0.2501 ± 0.0432 | 3.862 ± 3.827 |

Student's t-test between treatments (GL and GR CH), for each compartment and timepoint was completed. *indicates a statistical difference (p > 0.05)

TABLE 8
Peptide concentration after first pass metabolism of Genacol or Generic CH,
at every timepoint for the basolateral compartment. Student's t test between
treatments (GL and GR CH), for each compartment and timepoint was completed.

| | Basolateral T0 | | Basolateral T2 | | Basolateral T5 | |
|---|---|---|---|---|---|---|
| Treatment | GL | GR | GL | GR | GL | GR |
| Gly-Pro (μg/ml) | 0 ± 0 | 0 ± 0 | 12.56 ± 1.17 | 14.61 ± 1.03 | 13.49 ± 0.36 | 12.29 ± 1.11 |
| Hyp-Gly (μg/ml) | 0 ± 0 | 0 ± 0 | 0.4719 ± 0.0840 | 0.4513 ± 0.1998 | 0.2494 ± 0.0820 | 0.1839 ± 0.0427 |
| Ala-Hyp (μg/ml) | 0 ± 0 | 0 ± 0 | 1.826 ± 0.490 | 2.880 ± 0.631 | 5.848 ± 2.540 | 5.024 ± 1.849 |
| Pro-Hyp (μg/ml) | 0 ± 0 | 0 ± 0 | 1.980 ± 0.497 | 2.787 ± 0.164 | 2.768 ± 0.410 | 1.780 ± 0.300 |
| Gly-Pro-Hyp (μg/ml) | 0 ± 0 | 0 ± 0 | 1.206 ± 0.230* | N/A | 0.2483 ± 0.0228* | N/A |

*indicates a statistical difference (p > 0.05)

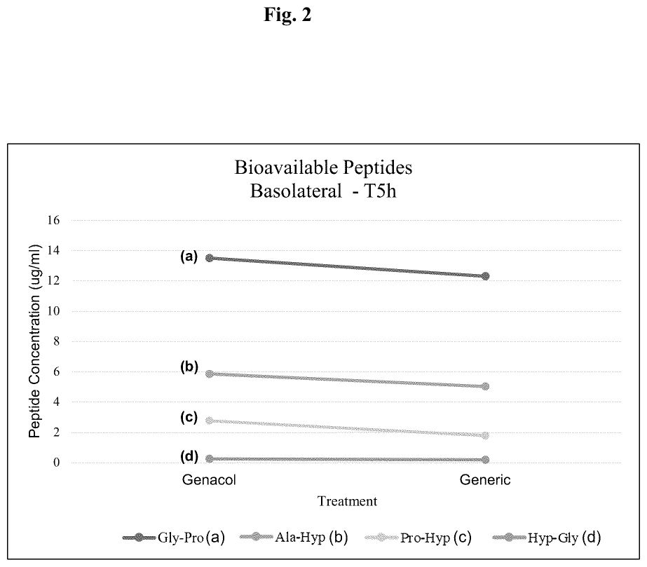

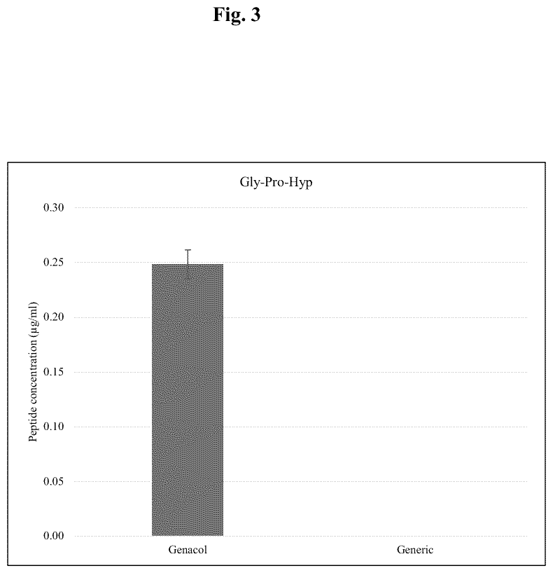

There were no significant differences between peptide concentrations between treatments for each compartment and timepoint (Tables 7 and 8, FIG. 2) except for the following: GR CH had a greater Ala-Hyp content (3.630±0.180 μg/ml) in the apical compartment after 2 h (during intestinal transport) than GL CH (0.3081±0.0748 μg/ml), although no difference was observed in the basolateral side at any point between treatments. Therefore, the relevance of this result is negligible. However, it is important to note that there was no detectable Gly-Pro-Hyp content after intestinal transport (basolateral side), as well as during the metabolism phase for the generic CH digested peptides. In contrast, GL CH had significant Gly-Pro-Hyp content at both timepoints (FIG. 3). Genacol CH is therefore more readily digested and metabolized into certain bioactive peptides, such as Gly-Pro-Hyp, as compared to Generic CH. Genacol CH has more Gly-Pro-Hyp that reaches the systemic circulation compared to the GR CH. The peptide profiles are different between Genacol and another bovine source CH (Generic brand). Furthermore, the bioavailability of key bioactive peptides is different between the two CHs products.

### Transport (%)

Transport (%) of CH peptides is calculated using basolateral T2/apical TO *100. All peptides were transported across the intestinal layer. Due to variability observed, no significant differences were observed between the transport of each peptide, except for Gly-Pro-Hyp, which was greater in GL CH treatment. There was no Gly-Pro-Hyp measured in GR CH (Table 9).

TABLE 9
The percentage of peptides that traveled
through the intestinal layer.

| Peptide (%) | GL | GR |
|---|---|---|
| Gly-Pro (μg/ml) | 33.11 ± 1.78 | 40.35 ± 1.65 |
| Hyp-Gly (μg/ml) | 62.41 ± 6.42 | 82.53 ± 21.09 |
| Ala-Hyp (μg/ml) | 9.27 ± 19.18 | 26.4 ± 24.15 |
| Pro-Hyp (μg/ml) | 19.18 ± 2.78 | 24.15 ± 0.82 |
| Gly-Pro-Hyp (μg/ml) | 59.44 ± 6.53* | N/A |

Student's t test between treatments (GL and GR CH), *indicates a statistical difference (p > 0.05)

### Biotransformation (%)

Biotransformation (%) of the CH peptides is calculated using basolateral T5/basolateral T2*100. As shown in Table 10, the liver cells metabolized more Pro-Hyp from the GL digesta than GR. A significantly greater upregulated transport/hepatic metabolism was seen with Genacol (˜151%) compared to the Generic (˜63%). Since there was no transportation of Gly-Pro-Hyp in the digestion of GR CH, there is no metabolism data. Biotransformation of other CH peptides by the GL CH compared to GR CH, were negligible and statistically insignificant.

TABLE 10
The percentage of CH peptides bio-transformed by the liver.

| Peptide (%) | GL | GR |
|---|---|---|
| Gly-Pro (μg/ml) | 109.2 ± 9.6 | 86.12 ± 14.09 |
| Hyp-Gly (μg/ml) | 55.16 ± 16.01 | 28.23 ± 6.55 |
| Ala-Hyp (μg/ml) | 304.9 ± 57.2 | 198.0 ± 107.6 |
| Pro-Hyp (μg/ml) | 151.4 ± 24.3* | 63.63 ± 8.63 |
| Gly-Pro-Hyp (μg/ml) | 22.32 ± 5.09* | 0 ± 0 |

Student's t test between treatments (GL and GR CH), *indicates a statistical difference (p > 0.05).

### Permeability (%)

Permeability can be represented as the percentage (%) of CH peptides that reach the systemic circulation after being ingested or added to a cell culture system. It is calculated as the portion of the initial dose, what is available after digestion (added to cells apical TO), over the peptide concentration measured from the basolateral side at T5.

All peptides reached the systemic circulation and had relatively high level of permeability (Table 11). Results indicated that no statistical difference in permeability was observed for all the peptides, except for Gly-Pro-Hyp which was only detected after GL CH peptide treatment (Table 11). The permeability for all the peptides investigated varied from 12.24±1.12-35.59±0.95% after GL CH peptide treatments, whereas the permeability range for the GR CH treatment was from 46.05±11.99-15.43±1.5%.

TABLE 11
Permeability of CH peptides,

| Treatment | GL | GR |
|---|---|---|
| Gly-Pro (μg/ml) | 35.59 ± 0.95 | 33.95 ± 3.07 |
| Hyp-Gly (μg/ml) | 32.99 ± 10.85 | 33.62 ± 7.81 |
| Ala-Hyp (μg/ml) | 29.69 ± 12.9 | 46.05 ± 16.95 |
| Pro-Hyp (μg/ml) | 26.81 ± 3.97 | 15.43 ± 2.6 |
| Gly-Pro-Hyp (μg/ml) | 12.24 ± 1.12* | N/A |

Student's t test between treatments (GL and GR CH) followed by TukeyHSD, *indicates a statistical difference (p > 0.05)

Permeability can depend on peptide length and amino acid composition. For instance, some peptides isolated from milk products have very low permeability such as LPYPY and WR, which are whey protein isolates that have a permeability of 0.05% and 0.47% respectively (Karaś, 2019; Lacroix, Chen, Kitts, & Li-Chan, 2017).

Furthermore, the B-casein peptide HLPLP has a limited permeability of 0.018%. Other milk derived peptides from casein having antioxidant properties have greater permeability; the permeability for the peptides IE, SDK and YPY are 44.81, 21.68 and 5.56% respectively. A milk hydrolysate showed only 7.8% permeability, whereas a non-specific permeability analysis of Hyp gelatin peptides from a rat study found that 41.91% of amino acid residues were absorbed in peptide form, although the analysis of individual peptides was not completed (Wang et al., 2015).

A previous study using Caco-2 cells and no hepatic cells, showed that only 3.59% of Gly-Pro-Hyp sourced from fish scales was transported across the cell monolayer (Sontakke, Jung, Piao, & Chung, 2016). In comparison, Genacol's CH showed a bioavailability of 59.44±6.53% for the peptide Gly-Pro-Hyp. This may be attributed to the physiologically relevant cell culture model used, or due to differences in collagen source material or processing methods. Regardless, this work demonstrates that the Genacol CHs product exhibits greater Gly-Pro-Hyp bioavailability compared to other products, both the generic CH tested from bovine source, and fish collagen from other publications.

### Antioxidant Capacity

Antioxidant capacity is recognized for its beneficial role in regulating heart disease, cancer and other diseases. Many patients look for products with greater antioxidant capacity due to their health promoting effects. Peptides have some antioxidant capacity.

There was an initial antioxidant capacity difference in the digesta between CH treatments using fluorescence recovery after photobleaching (FRAP) analysis (see FIG. below). Immediately, after digestion, the antioxidant capacity of GL was greater than GR (before being administered to the cell culture system).

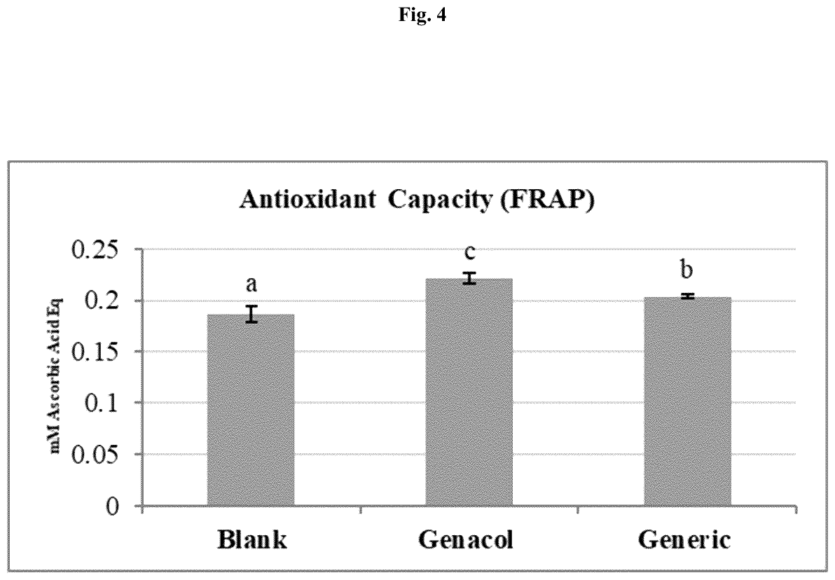

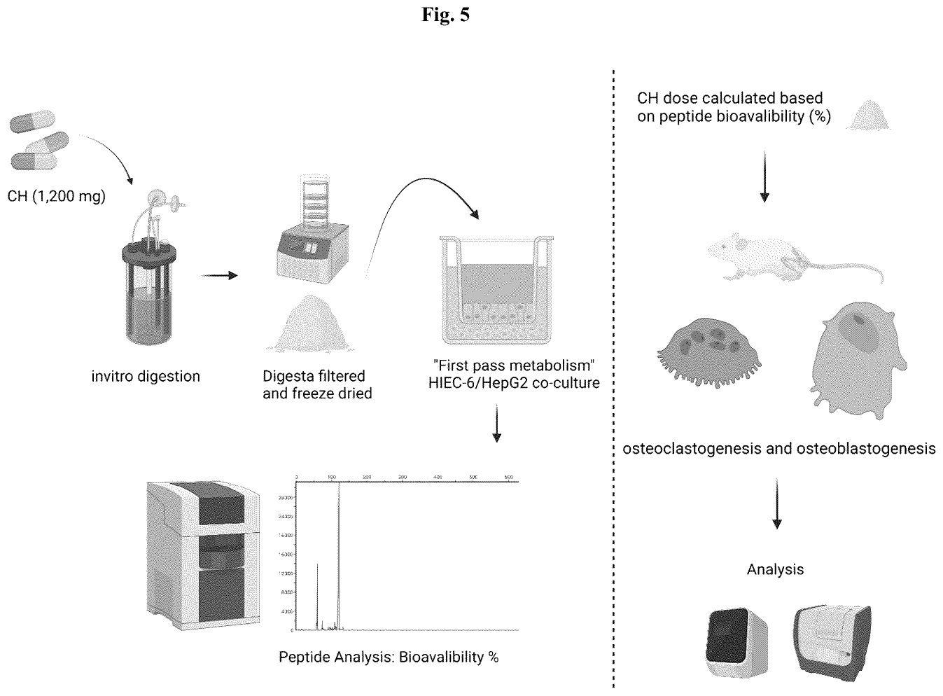

There were no significant differences in antioxidant capacity after peptide absorption; the antioxidant capacity of the peptides reaching the systemic circulation does not differ between Genacol CH or the Generic CH treatments (FIG. 4). Furthermore, no statistical differences between treatments, compartment or timepoint were observed using DPPH analysis (data not shown). However, the antioxidant capacity at the level of the intestine can still provide health benefits to the patients. Antioxidants do not necessarily need to be absorbed.

### Analysis of Genacol and Generic CH Products

There are some unidentified peaks from the small intestine, as well as peptide profiles before and after digestion using MALDI. This confirms that Genacol CH has a different peptide compositing compared to the Generic CH. This was assessed before first pass metabolisms to ensure the products were different to begin with, before completing costly cell culture methods. Processing methods between collagen manufacturers are different. Processing methods impact the initial peptide profile of a product, and can have an impact on how the products are digested and absorbed.

### Example 3: Effect of CH-GL on Osteoclasts

### OC Differentiation

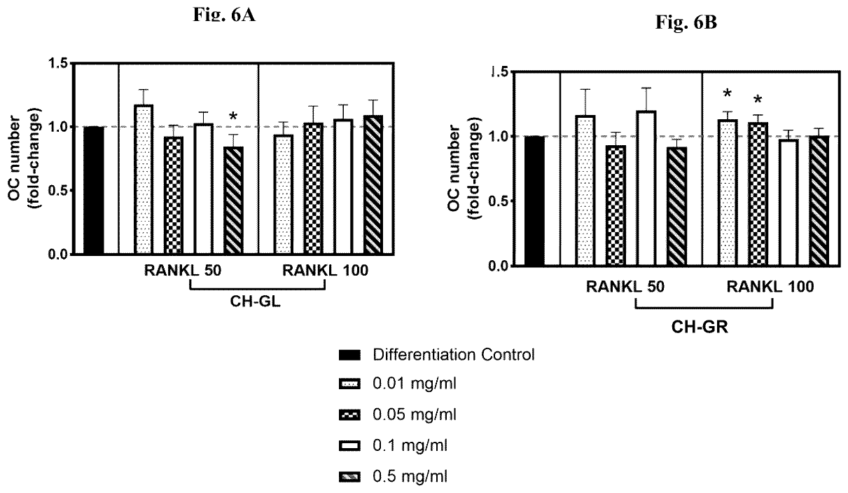

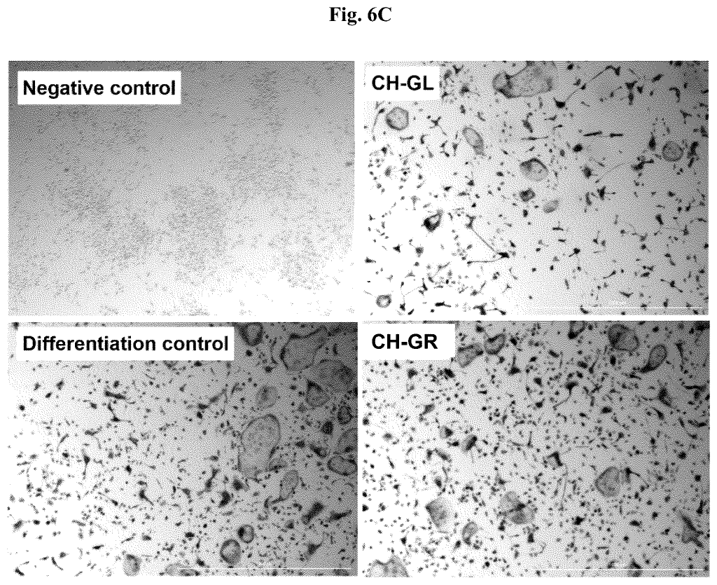

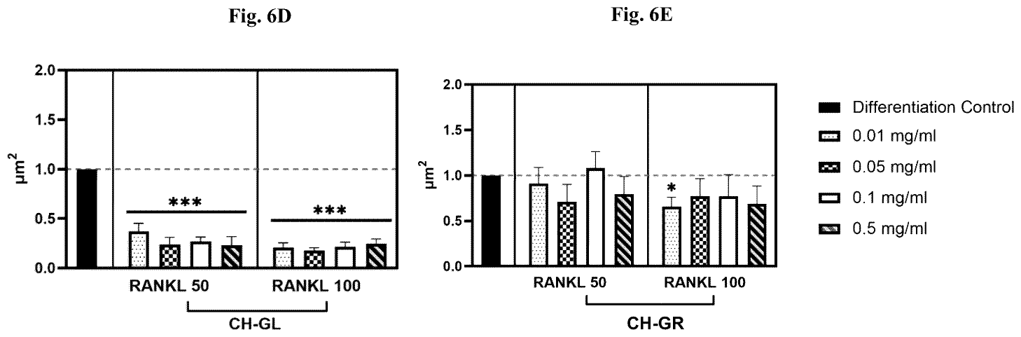

The Negative Control Showed No Positively (Purple) Stained OC, and No OC were observed for either CH control (FIG. 6C). Surprisingly, a 0.85-fold decrease in OC differentiation was observed with CH-GL treatment (0.05 mg/mL) with RANKL 50 ng/mL compared to the differentiation control (FIG. 6A). No other significant changes in differentiation for any CH-GL dose at either RANKL concentration were observed.

No significant changes in differentiation were observed after CH-GR treatments with RANKL 50 ng/mL. However, a significant increase in OC differentiation was observed at the higher RANKL dose (100 ng/mL) after CH-GR treatment (FIG. 6B). Differentiation was increased by 1.13-fold and 1.11-fold with CH-GR doses 0.01 and 0.05 mg/mL respectively, compared to the differentiation control.

### OC Size

Interestingly, the average OC size significantly decreased after CH-GL treatment (all doses) for both RANKL-initiated osteoclast differentiation concentrations, compared to control (FIG. 6D). In contrast, the average OC size was not significantly different compared to control after CH-GR treatments, except for 0.01 mg/mL with RANKL 100 ng/mL which was lower than control.

### OC Gene Expression

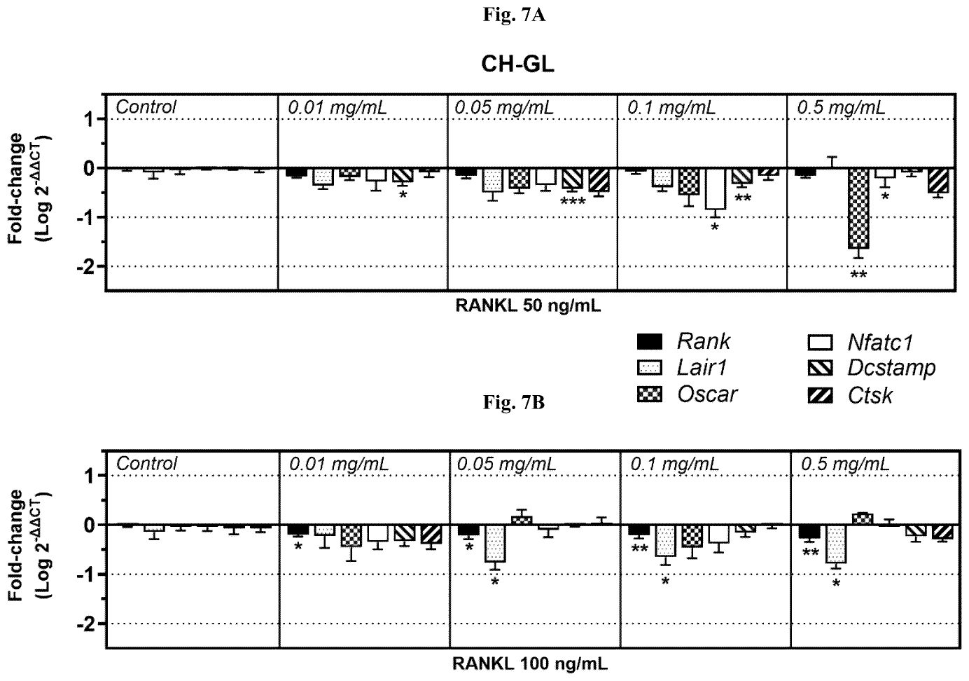

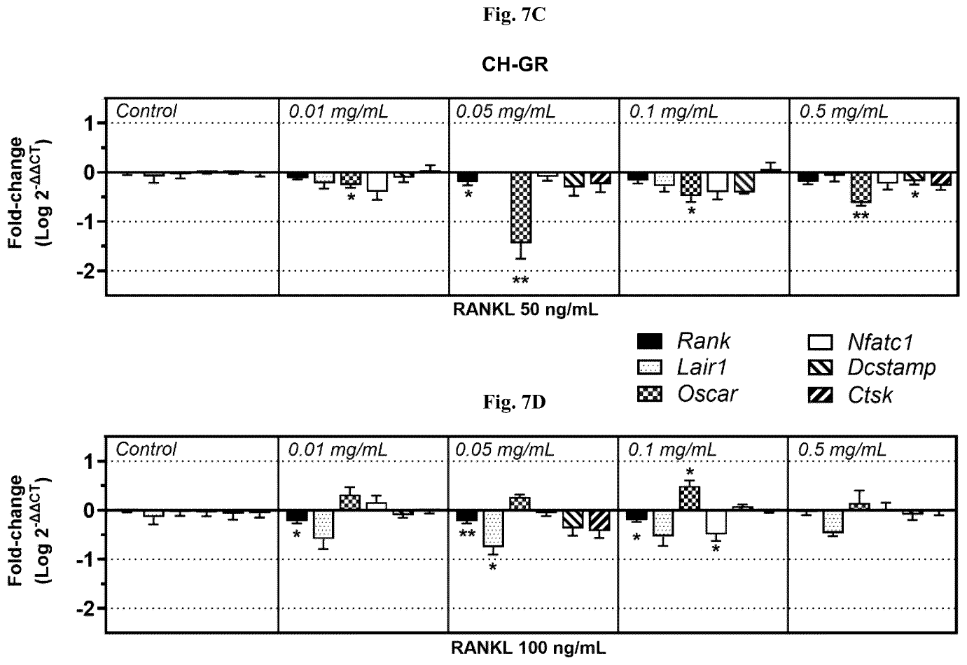

As RANKL expression by OCs can vary, investigating variable RANKL concentrations was considered. RANKL 50 ng/mL (FIGS. 7A and 7C) and 100 ng/mL (FIGS. 7B and 7D) were assessed. OC gene expression was affected by CH, although depended on CH treatment and RANKL dose (FIG. 7).

DC-stamp expression was significantly lower compared to control after CH-GL (0.01, 0.05 and 0.01 mg/mL) with RANKL 50 ng/mL (FIG. 7A). Fold decreases in DC-stamp expression were similar across CH-GL doses, however, the largest CH dose (0.5 mg/mL) showed no significant decrease or increase compared to control; a threshold could have been reached. Also, with RANKL 50 ng/mL, Nfactc1 expression was decreased by 0.8-fold but only after 0.1 mg/mL CH-GL. Oscar expression decreased with greater CH-GL doses, although was only significantly lower than control at 0.5 mg/mL.

Changes in gene expression with a greater RANKL dose (100 ng/mL) was also observed (FIG. 7B). However, the genes affected in response to CH doses varied compared to CH-GL at the lower RANKL dose. Notably RANK gene expression was not affected by CH-GL at lower RANKL doses, but with RANKL 100 ng/mL, RANK expression was decreased for all CH-GL doses. Lair-1 expression, which was also not affected by lower RANKL doses, showed decreased expression with a greater RANKL after every CH-GL dose, except for 0.01 mg/mL.

Oscar expression was decreased compared to control with each CH-GR dose and with RANKL 50 ng/mL. The only other change in gene expression with CH-GR and RANKL 50 ng/mL was a 0.18-fold decrease in DC-stamp (0.5 mg/mL).

As with CH-GL, no change in RANK or Lair-1 expression was observed with lower RANKL and CH-GR (FIG. 7C). However, with a greater RANKL (100 ng/mL), RANK expression was decreased after CH-GR (except for 0.5 mg/mL), and a 0.76-fold decrease in Lair-1 after 0.05 mg/mL was observed (FIG. 7D). An increase in Oscar was observed for each CH-GR dose with RANKL 100 ng/mL, but was only significantly greater than control with 0.1 mg/mL CH-GR.

No effects on Cathepsin K were observed for either CH treatment, regardless of dose or RANKL.

### Example 4: Effect of CH-GL on Osteoblasts

### OB Staining

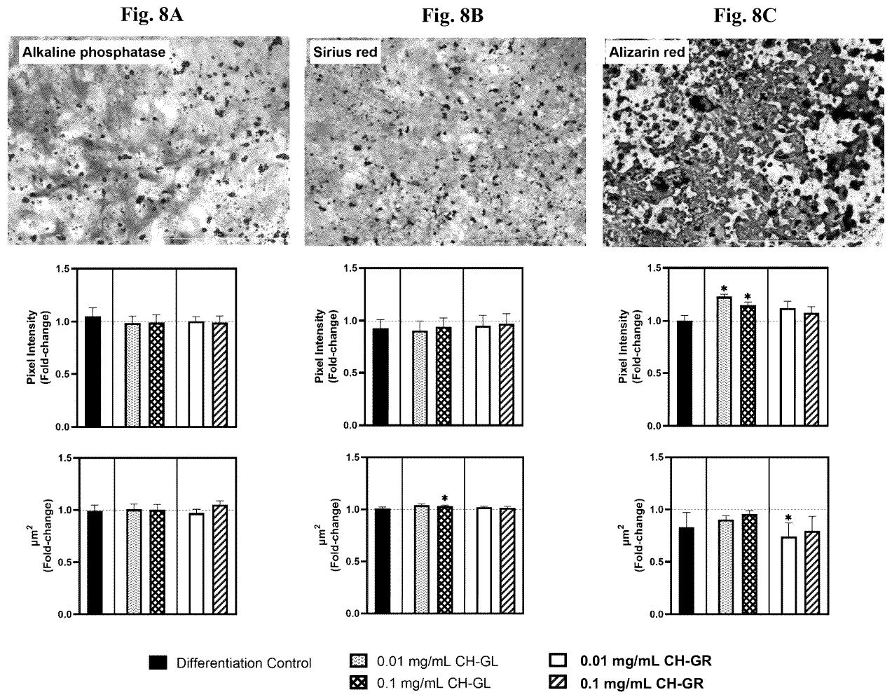

No effects on Cathepsin K were observed for either CH treatment, regardless of dose or RANKL. No change in pixel intensity or area was observed with alkaline phosphatase for either CH treatment, regardless of dose (FIG. 8A). In contrast, a 1.03-fold increased in area (μm²) was observed after CH-GL (0.1 mg/mL) compared to control with Sirius red staining. No changes in pixel intensity or area were observed after CH-GR with Sirius red (FIG. 8B).

The pixel intensity of alizarin red stain for both doses of CH-GL significantly increased compared to control (FIG. 8C). A 1.23-fold increase was observed after 0.01 mg/mL CH-GL compared to control, and a 1.15-fold increase for 0.1 mg/mL. No change in pixel intensity was observed after CH-GR for alizarin red, however, a 0.741-fold decrease in stained area compared to control was observed after 0.01 mg/mL CH-GR.

### OB Gene Expression

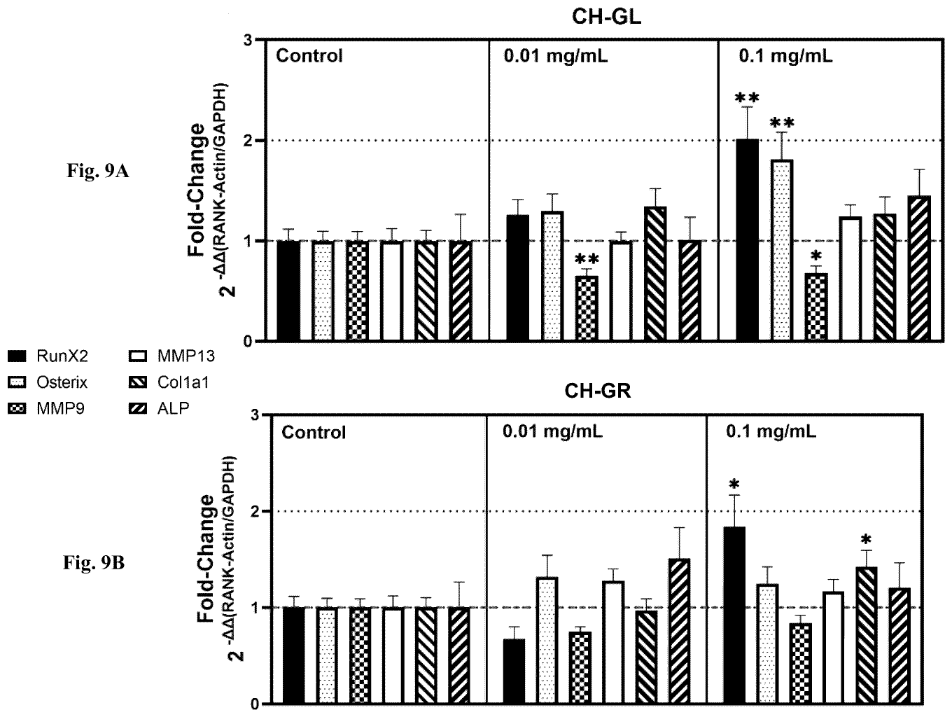

A few differences in osteoblastic gene expression were affected by CH treatment (FIG. 9). Gene expression for Runx2 and Osterix was increased by 2- and 1.8-fold, respectively with 0.1 mg/mL CH-GL (FIG. 9A). A decrease in MMP9 was observed with both doses of CH-GL.

As with CH-GL, Runx2 expression was also increased after 0.1 mg/mL CH-GR. However, no other changes in gene expression were observed for CH-GR, other than a 1.4-fold increased in Col1a1 at 0.1 mg/mL (FIG. 9B).

### Example 5: Discussion

A 2016 publication investigated the blood content and urinary excretion of peptides after collagen tripeptide ingestion in a human clinical trial (Yamamoto, Deguchi, Onuma, Numata, & Sakai, 2016). To create this collagen tripeptide product, Jellice Co., hydrolyzed collagen specifically at every third peptide bond following a Gly residue, thereby making a hydrolysate comprising of mainly Gly-X-Y tripeptides. More specifically, the collagen products were prepared from porcine skin collagen which were digested with a collagenase-type protease (Protease N, Nagase Chemtex Corporation, Osaka, Japan), then deionized with an ion exchange resin (DIAION, Mitsubishi Chemical, Tokyo, Japan) and passed through a 0.2-μm filter. Depending on the purity of the collagen product used, the tripeptide fraction was isolated with reversed-phase (RP)-HPLC. Di- or tripeptides are more easily absorbed though the intestinal cell wall barrier compared to larger molecular weight peptides. Yamamoto et al., found that the body can efficiently absorb and process peptides such as the repeating motive Gly-Pro-Hyp (such as gly-pro-hyp-gly-pro-hyp-gly-pro-hyp-gly-pro-hyp-), if they are already in a hydrolysate form. By observing a large content of Gly-Pro-Hyp in urinary excretion after oral administration, Yamamoto et al. showed that this peptide is relatively stable throughout digestion, absorption and passage through the blood stream. Some of the tripeptides hydrolyzed were also degraded into dipeptides such as Gly-Pro as well as Pro-Hyp, and Hyp-Gly. These peptides may be found in significant amounts.

The key differences between this study and the current investigation is that the source of collagen is different (porcine vs bovine), as well as the tripeptide component in Genacol's product was not specifically isolated. Furthermore, the content of tripeptide ingested in the clinical study compared to the content detected in the blood was very low, although no bioavailability data was calculated. Yamamoto et al., administered 80 mg/kg body weight of a collagen product dissolved in 100 mL of water to human participants, where the average intake of Gly-Pro-Hyp in each sample was 5682 μmol. Afterwards, the greatest Gly-Pro-Hyp content found in the blood was ˜22 uM peaking after 1 h. The ratio of administered peptide compared to the greatest detectable content of the peptide in the circulation, indicates an approximate bioavailability of only 0.387%, compared to Genacol's tripeptide bioavailability of 12.24±0.65% after 5 h.

The main sequence of collagen contains a glycine repeated every three amino acid residues (Gly-X-Y, where X and Y are amino acids). The most abundant sequence found in collagen is the repeative motif Gly-Pro-Hyp. This peptide has been established as having bioactive functions such as interacting with platelets as well as the central nervous system (Yamamoto et al., 2016). Additionally, this repeating Gly-X-Y sequence from collagen hydrolysates has been shown to promote bone healing and decreases atherosclerotic plaques. After absorption, the peptide Gly-Pro is produced by the breakdown of Gly-Pro-X peptides. The sequence Gly-Pro-X such as Gly-Pro-Hyp also played an important role as antioxidative peptides (Ao & Li, 2012).

The current work demonstrates that the bioactive peptides Pro-Hyp, Gly-Pro, Hyp-Gly, and Ala-Hyp and Gly-Pro-Hyp were able to undergo first pass metabolism. No statistical difference between any peptides measured between Genacol and Generic was observed, except for Gly-Pro-Hyp, which was significantly greater in Genacol after first pass metabolism. This trend reflects previous results obtained after in vitro digestion. We can assume that all the dipeptides are available in the blood stream to travel throughout the body and exert multiple bioactive functions. The biological threshold of activity of these peptides is not fully known. This threshold has been investigated in subsequent studies on bone and joint cultures, using the cell culture material produced from first pass metabolism, representing the peptide concentration available after digestion and absorption. In addition, liver cells metabolized more Pro-Hyp from Genacol's CH product compared to the Generic CH.

The antioxidant capacity of the digests from the small intestine were greater after Genacol CH digestion compared to the Generic CH product. The antioxidant capacity at the level of the intestinal can be beneficial and positively impact gastrointestinal health of a patient. Further breakdown of the bioactive peptides containing proline could impact the antioxidant potential as the peptides travel throughout the gastrointestinal system and eventually get degraded. This is primarily due to Proline having multiple bioactive functions such as regulating gene expression and cell differentiation, but also as a strong scavenger of oxidants (Wu et al., 2011). There was no difference in antioxidant capacity after absorption and metabolism of the peptides. The antioxidant contribution of the CHs at the level of the systemic circulation is therefore negligible, although as mentioned beforehand, the breakdown of the bioactive peptides containing proline could continue to impact the antioxidant potential as the peptides travel throughout the body and get further digested after reaching specific tissues.

Significant differences in bone cell activity were observed using CH doses based on bioavailability studies. Genacol's CH showed improved bone cell profiles: osteoclast activity and size were decreased, whereas osteoblast activity was slightly improved. Smaller osteoclasts are indicative of less active cells; therefore, less bone degradation occurs after Genacol treatment. Genacol, not the Generic collagen treatment, showed a decrease in MMP9 function in osteoblasts; this gene activates cytokines which regulate tissue remolding as well as enzymes that degrade the extracellular matrix. The Generic CH showed an increase in osteoclast differentiation with no major changes to osteoblasts cell activity. An induction of osteoclast differentiation after the Generic treatment indicates the potential for greater bone degradation. These data highlight the physiological significance of CH peptides after digestion of Genacol CH, as compared to generic CH. These results explain why Genacol continues to demonstrate positive clinical results, and may aid in the treatment of certain diseases such as osteoarthritis, or diseases mediated by osteoclasts.

The present description refers to a number of documents, the content of which is herein incorporated by reference in their entirety.

The scope of the claims should not be limited by the preferred embodiments set forth in the examples, but should be given the broadest interpretation consistent with the description as a whole. When a range of values is described, the person of ordinary skill in the art would understand that all values within this range are included, also not specifically listed.

In some aspects, embodiments of the present invention as described herein include the following items:
1. A composition comprising one or more bioactive peptides from collagen hydrolysate and a pharmaceutically acceptable excipient.
2. The composition of item 1, wherein the one or more peptides comprise 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids.
3. The composition of item 1, wherein the one or more peptides comprise a dipeptide or tripeptide.
4. The composition of any one of items 1 to 3, wherein the one or more peptides comprise a combination or permutation of Hyp, Gly, Pro, and/or Ala.
5. The composition of any one of items 1 to 4, wherein the one or more peptides comprise Ala-Ala, Ala-Gly, Ala-Hyp, Ala-Pro, Gly-Gly, Gly-Hyp, Gly-Pro, Hyp-Hyp, Hyp-Pro, or Pro-Pro.
6. The composition of any one of items 1 to 4, wherein the one or more peptides comprise Ala-Ala-Ala, Ala-Ala-Gly, Ala-Ala-Hyp, Ala-Ala-Pro, Ala-Gly-Ala, Ala-Gly-Gly, Ala-Gly-Hyp, Ala-Gly-Pro, Ala-Hyp-Ala, Ala-Hyp-Gly, Ala-Hyp-Hyp, Ala-Hyp-Pro, Ala-Pro-Ala, Ala-Pro-Gly, Ala-Pro-Hyp, Ala-Pro-Pro, Gly-Gly-Ala, Gly-Gly-Gly, Gly-Gly-Hyp, Gly-Gly-Pro, Gly-Hyp-Ala, Gly-Hyp-Gly, Gly-Hyp-Hyp, Gly-Hyp-Pro, Gly-Pro-Ala, Gly-Pro-Gly, Gly-Pro-Hyp, Gly-Pro-Pro, Hyp-Hyp-Ala, Hyp-Hyp-Gly, Hyp-Hyp-Hyp, Hyp-Hyp-Pro, Hyp-Pro-Ala, Hyp-Pro-Gly, Hyp-Pro-Hyp, Hyp-Pro-Pro, Pro-Pro-Ala, Pro-Pro-Gly, Pro-Pro-Hyp, or Pro-Pro-Pro.
7. The composition of any one of items 1 to 4, wherein the one or more peptides comprise Ala-Hyp, Pro-Hyp, Hyp-Gly, Gly-Pro, and/or Gly-Pro-Hyp.
8. The composition of any one of items 1 to 5, wherein the one or more peptides comprise Pro-Hyp-Gly, Pro-Gly-Hyp, Gly-Ala-Hyp, Ala-Cys-Ser, Glu-Asp, Gly-Gln, Leu-Hyp, Met-Leu, Phe-Pro, Pro-Gly-Leu, Pro-Leu, Ser-Gly-Pro, Ser-Hyp, Ser-Pro, Thr-Tyr, Val-Ala, and/or Gly-Pro-Ala.
9. The composition of any one of items 1 to 8, further comprising a diluent, carrier, gelatin, microcrystalline cellulose, silicon dioxide, vegetable magnesium stearate, magnesium stearate, caramel, Citric acid, Glycine, L-Histidine, L-Lysine, L-Methionine, L-isoleucine, leucine, L-phenylalanine, potassium sorbate, purified water, sodium benzoate, sodium citrate, Stevia, natural vanilla flavor, flavor, aroma, and/or a compound improving taste and/or odor.
10. The composition of any one of items 1 to 9, further comprising hyaluronic acid, amino acid reissued such as the amino acid GABA, glucosamine, melatonin, MSM, chondroitin, vitamins such as vitamin C, curcuma and/or curcumin.
11. The composition of any one of items 1 to 10, wherein the composition is in a solid, gel, or liquid form.
12. The composition of any one of items 1 to 11, wherein the collagen hydrolysate is prepared from beef, pork, poultry, or fish skins or scales, preferably from beef or pork.
13. The composition of any one of items 1 to 12, wherein the collagen is from skin, hides, or bone.
14. The composition of any one of items 1 to 13, wherein the composition is a pharmaceutical or nutraceutical composition.
15. The composition of any one of items 1 to 14, wherein the collagen hydrolysate has no bitter taste or odor.
16. A composition comprising one or more bioactive peptides from collagen hydrolysate and a pharmaceutically acceptable excipient, wherein the one or more bioactive peptides comprise the tripeptide Gly-Pro-Hyp.
17. The composition of item 16, wherein the one or more bioactive peptides further comprise of a dipeptide selected from the group consisting of Gly-Pro, Hyp-Gly, Ala-Hyp, Pro-Hyp, and any combination thereof.
18. A composition as defined in any one of items 1 to 17, for use in preventing and/or reducing joint pain in a patient.

19. Use of the composition as defined in any one of items 1 to 17, for preventing and/or reducing joint pain in a patient.

20. Use of the composition as defined in any one of items 1 to 17, for the manufacture of a medicament for preventing and/or reducing joint pain in a patient.

21. The composition for use of item 18 or the use of item 19 or 20, wherein the joint pain is shoulder, elbow, hand, lumbar spine, hip or knee pain.

22. A composition as defined in any one of items 1 to 17, for use in the treatment and/or prevention of arthritis in a patient.
23. Use of the composition as defined in any one of items 1 to 17, for the treatment and/or prevention of arthritis in a patient.
24. Use of the composition as defined in any one of items 1 to 17, for the manufacture of a medicament for the treatment and/or prevention of arthritis in a patient.
25. The composition for use of item 22 or the use of item 23 or 24, wherein the arthritis is osteoarthritis.
26. A composition as defined in any one of items 1 to 17, for use in the treatment and/or prevention of an osteoclast-related disease or disorder in a patient.
27. Use of the composition as defined in any one of items 1 to 17, for the treatment and/or prevention of an osteoclast-related disease or disorder in a patient.
28. Use of the composition as defined in any one of items 1 to 17, for the manufacture of a medicament for the treatment and/or prevention of an osteoclast-related disease or disorder in a patient.
29. The composition for use of item 26 or the use of item 27 or 28, wherein the osteoclast-related disease or disorder is selected from the group consisting of osteoporosis, osteoarthritis, rheumatoid arthritis, Paget's Bone Disease, bone tumors, periprosthetic osteolysis, osteopetrosis, osteopenia, or osteoclastoma.
30. A composition as defined in any one of items 1 to 17, for use in inhibiting the activity and/or expression of osteoclasts.
31. Use of the composition as defined in any one of items 1 to 17, for inhibiting the activity and/or expression of osteoclasts.
32. Use of the composition as defined in any one of items 1 to 17, for the manufacture of a medicament for inhibiting the activity and/or expression of osteoclasts.
33. A composition as defined in any one of items 1 to 17, for use in increasing the activity and/or expression of osteoblasts.
34. Use of the composition as defined in any one of items 1 to 17, for increasing the activity and/or expression of osteoblasts.
35. Use of the composition as defined in any one of items 1 to 17, for the manufacture of a medicament for increasing the activity and/or expression of osteoblasts.
36. A method for preventing and/or reducing joint pain in a patient, said method comprising administering the composition as defined in any one of items 1 to 17 to said patient.
37. The method of item 36, wherein the joint paint wherein the joint pain is shoulder, elbow, hand, lumbar spine, hip or knee pain.
38. A method for treating and/or preventing arthritis in a patient, said method comprising administering the composition as defined in any one of items 1 to 17 to said patient.
39. The method of item 38, wherein the arthritis is osteoarthritis. 40. A method for treating and/or preventing an osteoclast-related disease or disorder in a patient, said method comprising administering the composition as defined in any one of items 1 to 17 to said patient.
41. The method of item 40, wherein the osteoclast-related disease or disorder is selected from the group consisting of osteoporosis, osteoarthritis, rheumatoid arthritis, Paget's Bone Disease, bone tumors, periprosthetic osteolysis, osteopetrosis, osteopenia, or osteoclastoma.
42. A method for inhibiting the activity and/or expression of osteoclasts, said method comprising treating osteoclasts with the composition as defined in any one of items 1 to 17.
43. A method for increasing the activity and/or expression of osteoblasts, said method comprising treating osteoblasts with the composition as defined in any one of items 1 to 17.
44. The method of item 42 or 43, wherein the method is performed in vitro, ex vivo, or in vivo.

### REFERENCES

Albenzio, M., Santillo, A., Caroprese, M., Malva, A. d., & Marino, R. (2017). Bioactive Peptides in Animal Food Products. Foods, 6(5), 35.
Alemán, A., Gómez-Guillén, M. C., & Montero, P. (2013). Identification of ace-inhibitory peptides from squid skin collagen after in vitro gastrointestinal digestion. Food Research International, 54(1), 790-795.
Ao, J., & Li, B. (2012). Amino acid composition and antioxidant activities of hydrolysates and peptide fractions from porcine collagen. Food Science and Technology International, 18(5), 425-434.
Bello, A. E., & Oesser, S. (2006). Collagen hydrolysate for the treatment of osteoarthritis and other joint disorders: a review of the literature. Current Medical Research & Opinion, 22(11), 2221-2232.
Bernado, M. L. R., & Azarcon, J., A. C. (2012). A randomized control trial on the effects of oral collagen treatment on the medial knee joint space and functional outcome among Veterans Medical Center patients diagnosed with osteoarthritis of the knee. Philippine Academy of Rehabilitation Medicine Processings 4, 1-8.
Boraschi-Diaz, I.; Komarova, S. V. The protocol for the isolation and cryopreservation of osteoclast precursors from mouse bone marrow and spleen. Cytotechnology 2016, 68, 105-114, doi:10.1007/s10616-014-9759-3.
Bruyère, O., Zegels, B., Leonori, L., Rabenda, V., Janssen, A., Bourges, C., & Reginster, J. Y. (2012). Effect of collagen hydrolysate in articular pain: A 6-month randomized, double-blind, placebo controlled study. Complementary Therapies in Medicine, 20(3), 124-130.
Castañeda, S.; Vicente, E. F. Osteoarthritis: More than cartilage degeneration. Clin Rev Bone Miner Metab 2017, 15, 69-81, doi:10.1007/s12018-017-9228-6.
Ekbatan, S. S., Iskandar, M. M., Sleno, L., Sabally, K., Khairallah, J., Prakash, S., and Kubow. S. (2018). Absorption and Metabolism of Phenolics from Digests of Polyphenol-Rich Potato Extracts Using the Caco-2/HepG2 Co-Culture System. Foods, 7(8), 1-11.
Feliciano, D. D. S., Gonzalex-Suarez, C. B., Bernardo-Bueno, M. M., Malvar, A. K. G., Cua, R. C. A., Tan-Sales, B. G. K., Aycardo, S. M. O., Tan-Ong, M., Chan, R., & Reyes, F. D. L. (2017). Effect of collagen hydrolysate as adjuvant treatment to exercise for knee osteoarthritis. Philippine Academy of Rehabilitation Medicine, 9(1).
Ferraro, V., Anton, M., & Santé-Lhoutellier, V. (2016). The “sisters” α-helices of collagen, elastin and keratin recovered from animal by-products: Functionality, bioactivity and trends of application. Trends in Food Science & Technology, 51, 65-75.
Hatanaka, T., K. Kawakami, and M. Uraji. (2014). Inhibitory effect of collagen-derived tripeptides on dipeptidylpeptidase-IV activity. Journal of Enzyme Inhibition and Medicinal Chemistry. 29(6), p. 823-828.
Iskandar, M., Lands, L., Sabally, K., Azadi, B., Meehan, B., Mawji, N., Skinner, C., & Kubow, S. (2015). High hydrostatic pressure pretreatment of whey protein isolates improves their digestibility and antioxidant capacity. Foods, 4(2), 184.
Iwai, K., et al., (2005). Identification of food-derived collagen peptides in human blood after oral ingestion of gelatin hydrolysates. Journal of Agricultural and Food Chemistry. 53(16), p. 6531-6536.
Karaś, M. (2019). Influence of physiological and chemical factors on the absorption of bioactive peptides. International Journal of Food Science & Technology, 54(5), 1486-1496.
Knight, C. G., et al., (1999). Collagen-platelet interaction: Gly-Pro-Hyp is uniquely specific for platelet Gp VI and mediates platelet activation by collagen. Cardiovascular Research. 41(2), p. 450-457.
Kumar, S., Sugihara, F., Suzuki, K., Inoue, N., & Venkateswarathirukumara, S. (2015). A double-blind, placebo-controlled, randomised, clinical study on the effectiveness of collagen peptide on osteoarthritis. Journal of the Science of Food and Agriculture, 95(4), 702-707.
Lacroix, I. M. E., Chen, X. M., Kitts, D. D., & Li-Chan, E. C. Y. (2017). Investigation into the bioavailability of milk protein-derived peptides with dipeptidyl-peptidase IV inhibitory activity using Caco-2 cell monolayers. Food Funct, 8(2), 701-709. doi:10.1039/c6fo01411a
Löfvall, H., Newbould. H., Karsdal, M. A., Dziegiel, M. H., Richter, J., Henriksen, K and Thudium, C. S. Osteoclasts degrade bone and cartilage knee joint compartments through different resorption processes. Arthritis Research & Therapy (2018) 20:67
Louati, K., et al. (2015). Association between diabetes mellitus and osteoarthritis: systematic literature review and meta-analysis. RMD Open, 1(1), p. e000077.
Miranda, L., Deusser, H., & Evers, D. (2013). The impact of in vitro digestion on bioaccessibility of polyphenols from potatoes and sweet potatoes and their influence on iron absorption by human intestinal cells. Food & Function, 4(11), 1595-1601.
Nakatani, S., et al., (2009). Chondroprotective effect of the bioactive peptide prolyl-hydroxyproline in mouse articular cartilage in vitro and in vivo. Osteoarthritis and Cartilage, 17(12), p. 1620-1627.
Oesser, S., Adam, M., Babel, W., & Seifert, J. (1999). Oral administration of 14C labeled gelatin hydrolysate leads to an accumulation of radioactivity in cartilage of mice (C57/BL). The Journal of Nutrition, 129(10), 1891-1895.
Oesser, S., & Seifert, J. (2003). Stimulation of type II collagen biosynthesis and secretion in bovine chondrocytes cultured with degraded collagen. Cell and Tissue Research, 311(3), 393-399.
Orriss, I. R.; Hajjawi, M. O. R.; Huesa, C.; MacRae, V. E.; Arnett, T. R. Optimisation of the differing conditions required for bone formation in vitro by primary osteoblasts from mice and rats. Int J Mol Med 2014, 34, 1201-1208, doi:10.3892/ijmm.2014.1926.
Orriss, I. R.; Taylor, S. E.; Arnett, T. R. Rat osteoblast cultures. Methods Mol Biol 2012, 816, 31-41, doi:10.1007/978-1-61779-415-5_3.
Piva, S. R., et al. (2015). Links between osteoarthritis and diabetes: implications for management from a physical activity perspective. Clinics in Geriatric Medicine. 31(1), p. 67-viii.
Schrieber et al. (2007). The role of collagen hydrolysate in the prophylaxis of osteoarthritis and osteoporosis, in Gelatin handbook, Wiley ed, pp. 301-309
Sontakke, S. B., Jung, J. H., Piao, Z., & Chung, H. J. (2016). Orally Available Collagen Tripeptide: Enzymatic Stability, Intestinal Permeability, and Absorption of Gly-Pro-Hyp and Pro-Hyp. J Agric Food Chem, 64(38), 7127-7133. doi:10.1021/acs.jafc.6b02955
Takenaka, T., Harada, N., Kuze, J., Chiba, M., Iwao, T., & Matsunaga, T. (2014). Human small intestinal epithelial cells differentiated from adult intestinal stem cells as a novel system for predicting oral drug absorption in humans. Drug Metabolism and Disposition, 42(11), 1947-1954.
Takenaka, T., Harada, N., Kuze, J., Chiba, M., Iwao, T., & Matsunaga, T. (2016). Application of a Human Intestinal Epithelial Cell Monolayer to the Prediction of Oral Drug Absorption in Humans as a Superior Alternative to the Caco-2 Cell Monolayer. Journal of Pharmaceutical Sciences 105, 915-924.
Wang, L., Wang, Q., Liang, Q., He, Y., Wang, Z., He, S., Xu, J., & Ma, H. (2015). Determination of bioavailability and identification of collagen peptide in blood after oral ingestion of gelatin. Journal of the Science of Food and Agriculture, 95(13), 2712-2717.
Wauquier, F., Daneault, A., Granel, H., Prawitt, J., Fabien Soulé, V., Berger, J., Pereira, B., Guicheux, J., Rochefort, G. Y., Meunier, N., Blot, A., and Wittrant, Y. (2019). Human Enriched Serum Following Hydrolysed Collagen Absorption Modulates Bone Cell Activity: from Bedside to Bench and Vice Versa. Nutrients 11(1249), 1-17,
Wu, G., Bazer, F. W., Burghardt, R. C., Johnson, G. A., Kim, S. W., Knabe, D. A., Li, P., Li, X., McKnight, J. R., Satterfield, M. C., & Spencer, T. E. (2011). Proline and hydroxyproline metabolism: implications for animal and human nutrition. Amino acids, 40(4), 1053-1063. doi:10.1007/s00726-010-0715-z
Yamamoto, S., Deguchi, K., Onuma, M., Numata, N., & Sakai, Y. (2016). Absorption and urinary excretion of peptides after collagen tripeptide ingestion in humans. Biological & Pharmaceutical Bulletin, 39(3), 428-434.

1-44. (canceled)

45. A composition comprising one or more bioactive peptides from collagen hydrolysate and a pharmaceutically acceptable excipient, wherein the one or more bioactive peptides comprise the tripeptide Gly-Pro-Hyp.

46. The composition of claim 45, wherein the one or more bioactive peptides further comprise:
a) 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids;
b) a dipeptide or tripeptide; and/or
c) a combination or permutation of Hyp, Gly, Pro, and/or Ala.

47. The composition of claim 45, wherein the one or more peptides comprise Ala-Ala, Ala-Gly, Ala-Hyp, Ala-Pro, Gly-Gly, Gly-Hyp, Gly-Pro, Hyp-Hyp, Hyp-Pro, Hyp-Gly, Pro-Hyp, and/or Pro-Pro.

48. The composition of claim 45, wherein the one or more bioactive peptides comprise Ala-Ala-Ala, Ala-Ala-Gly, Ala-Ala-Hyp, Ala-Ala-Pro, Ala-Gly-Ala, Ala-Gly-Gly, Ala-Gly-Hyp, Ala-Gly-Pro, Ala-Hyp-Ala, Ala-Hyp-Gly, Ala-Hyp-Hyp, Ala-Hyp-Pro, Ala-Pro-Ala, Ala-Pro-Gly, Ala-Pro-Hyp, Ala-Pro-Pro, Gly-Gly-Ala, Gly-Gly-Gly, Gly-Gly-Hyp, Gly-Gly-Pro, Gly-Hyp-Ala, Gly-Hyp-Gly, Gly-Hyp-Hyp, Gly-Hyp-Pro, Gly-Pro-Ala, Gly-Pro-Gly, Gly-Pro-Hyp, Gly-Pro-Pro, Hyp-Hyp-Ala, Hyp-Hyp-Gly, Hyp-Hyp-Hyp, Hyp-Hyp-Pro, Hyp-Pro-Ala, Hyp-Pro-Gly, Hyp-Pro-Hyp, Hyp-Pro-Pro, Pro-Pro-Ala, Pro-Pro-Gly, Pro-Pro-Hyp, and/or Pro-Pro-Pro.

49. The composition of claim 45, wherein the one or more bioactive peptides further comprise Ala-Hyp, Pro-Hyp, Hyp-Gly, and/or Gly-Pro.

50. The composition of claim 45, wherein the one or more bioactive peptides further comprise Pro-Hyp-Gly, Pro-Gly-Hyp, Gly-Ala-Hyp, Ala-Cys-Ser, Glu-Asp, Gly-Gln, Leu-Hyp, Met-Leu, Phe-Pro, Pro-Gly-Leu, Pro-Leu, Ser-Gly-Pro, Ser-Hyp, Ser-Pro, Thr-Tyr, Val-Ala, and/or Gly-Pro-Ala.

51. The composition of claim 45, further comprising a diluent, carrier, gelatin, microcrystalline cellulose, silicon dioxide, vegetable magnesium stearate, magnesium stearate, caramel, Citric acid, Glycine, L-histidine, L-lysine, L-methionine, L-isoleucine, leucine, L-phenylalanine, potassium sorbate, purified water, sodium benzoate, sodium citrate, stevia, natural vanilla flavor, flavor, aroma, and/or a compound improving taste and/or odor.

52. The composition of claim 45, further comprising hyaluronic acid, amino acid reissued such as the amino acid GABA, glucosamine, melatonin, MSM, chondroitin, vitamins such as vitamin C, curcuma and/or curcumin.

53. The composition of claim 45, wherein the composition is in a solid, gel, or liquid form.

54. The composition of claim 45, wherein the collagen hydrolysate is prepared from beef, pork, poultry, or fish skins or scales, preferably from beef or pork.

55. The composition of claim 45, wherein the collagen is from skin, hides, or bone.

56. The composition of claim 45, wherein the composition is a pharmaceutical or nutraceutical composition.

57. The composition of claim 45, wherein the collagen hydrolysate has no bitter taste or odor.

58. The composition of claim 45, wherein the one or more bioactive peptides further comprise Gly-Pro, Hyp-Gly, Ala-Hyp, and Pro-Hyp.

59. A method for preventing and/or reducing joint pain in a patient, for treating and/or preventing arthritis in a patient, or for treating and/or preventing an osteoclast-related disease or disorder in a patient, said method comprising administering a therapeutically-effective amount of the composition of claim 45 to said patient.

60. The method of claim 59, wherein the joint pain is shoulder, elbow, hand, lumbar spine, hip or knee pain.

61. The method of claim 59, wherein the arthritis is osteoarthritis.

62. The method of claim 59, wherein the osteoclast-related disease or disorder is selected from the group consisting of osteoporosis, osteoarthritis, rheumatoid arthritis, Paget's Bone Disease, bone tumors, periprosthetic osteolysis, osteopetrosis, osteopenia, and osteoclastoma.

63. A method for inhibiting the activity and/or expression of osteoclasts or for increasing the activity and/or expression of osteoblasts, said method comprising treating osteoclasts or osteoblasts with the composition of claim 45.

64. The method of claim 63, wherein the method is performed in vitro, ex vivo, or in vivo.


SEQUENCE LISTING (28 sequences)

SEQ ID NO: 1 - DNA, 20 bp, Artificial Sequence
  RANK forward primer
gcatcccttgcagctcaaca

SEQ ID NO: 2 - DNA, 20 bp, Artificial Sequence
  RANK reverse primer
atggaagagctgcagaccac

SEQ ID NO: 3 - DNA, 20 bp, Artificial Sequence
  Oscar forward primer
tcgctgatactccagctgtc

SEQ ID NO: 4 - DNA, 20 bp, Artificial Sequence
  Oscar reverse primer
tctggggagctgatccgtta

SEQ ID NO: 5 - DNA, 21 bp, Artificial Sequence
  Cathepsin K forward primer
cagtagccacgcttcctatcc

SEQ ID NO: 6 - DNA, 20 bp, Artificial Sequence
  Cathepsin K reverse primer
acgccgagagatttcatcca

SEQ ID NO: 7 - DNA, 20 bp, Artificial Sequence
  Lair-1 forward primer
ctgtacccctgggcaacttt

SEQ ID NO: 8 - DNA, 20 bp, Artificial Sequence
  Lair-1 reverse primer
ttccataaaggtgctgccgt

SEQ ID NO: 9 - DNA, 20 bp, Artificial Sequence
  NFATC1 forward primer
cccggagttcgacttcgatt

SEQ ID NO: 10 - DNA, 20 bp, Artificial Sequence
  NFATC1 reverse primer
tctctgtaggcttccaggct

SEQ ID NO: 11 - DNA, 20 bp, Artificial Sequence
  DC-STAMP forward primer
tttccacgaagccctagctg

SEQ ID NO: 12 - DNA, 20 bp, Artificial Sequence
  DC-STAMP reverse primer
gcgttcctaccttcacggag

SEQ ID NO: 13 - DNA, 20 bp, Artificial Sequence
  Col1a1 forward primer
gagcggagagtactggatcg

SEQ ID NO: 14 - DNA, 20 bp, Artificial Sequence
  Col1a1 reverse primer
gttcgggctgatgtaccagt

SEQ ID NO: 15 - DNA, 19 bp, Artificial Sequence
  Alkaline phosphatase forward primer
caggccgccttcataagca

SEQ ID NO: 16 - DNA, 20 bp, Artificial Sequence
  Alkaline phosphatase reverse primer
gtgccgatggccagtactaa

SEQ ID NO: 17 - DNA, 20 bp, Artificial Sequence
  RunX2 forward primer
gcttctcagctttagcgtcg

SEQ ID NO: 18 - DNA, 19 bp, Artificial Sequence
  RunX2 reverse primer
aaggtgccgggaggtaagt

SEQ ID NO: 19 - DNA, 20 bp, Artificial Sequence
  Osterix forward primer
gatggcgtcctctctgcttg

SEQ ID NO: 20 - DNA, 20 bp, Artificial Sequence
  Osterix reverse primer
gggctgaaaggtcagcgtat

SEQ ID NO: 21 - DNA, 20 bp, Artificial Sequence
  MMP9 forward primer
ccagccgacttttgtggtct

SEQ ID NO: 22 - DNA, 20 bp, Artificial Sequence
  MMP9 reverse primer
tggcctttagtgtctggctg

SEQ ID NO: 23 - DNA, 20 bp, Artificial Sequence
  MMP13 forward primer
gccattaccagtctccgagg

SEQ ID NO: 24 - DNA, 20 bp, Artificial Sequence
  MMP13 reverse primer
ggtcacgggatggatgttca

SEQ ID NO: 25 - DNA, 20 bp, Artificial Sequence
  Actin-B forward primer
tgttaccaactgggacgaca

SEQ ID NO: 26 - DNA, 20 bp, Artificial Sequence
  Actin-B reverse primer
ggggtgttgaaggtctcaaa

SEQ ID NO: 27 - DNA, 20 bp, Artificial Sequence
  GAPDH forward primer
acccagaagactgtggatgg

SEQ ID NO: 28 - DNA, 20 bp, Artificial Sequence
  GAPDH reverse primer
cacattgggggtaggaacac